(19) United States
(12) Patent Application Publication
(10) Pub. No.: US 2025/0115843 A1
(43) Pub. Date: Apr. 10, 2025

(54) CELL PRODUCTION CARTRIDGE AND CELL PRODUCTION APPARATUS

(71) Applicants: CANON MEDICAL SYSTEMS CORPORATION, Otawara-shi (JP); Canon Kabushiki Kaisha, Tokyo (JP)

(72) Inventors: Takehito YAMAGUCHI, Kawasaki (JP); Akane KUBOYAMA, Kawasaki (JP); Kazutaka YANAGITA, Yokohama (JP); Yosuke YAMAGUCHI, Kawasaki (JP)

(73) Assignees: CANON MEDICAL SYSTEMS CORPORATION, Otawara-shi (JP); Canon Kabushiki Kaisha, Tokyo (JP)

(21) Appl. No.: 18/905,895

(22) Filed: Oct. 3, 2024

(30) Foreign Application Priority Data
Oct. 6, 2023 (JP) 2023-174522
Oct. 2, 2024 (JP) 2024-173212

(51) Int. Cl. C12M 3/00; C12M 1/00

(52) U.S. Cl. CPC C12M 23/42; C12M 23/40; C12M 29/06

(57) ABSTRACT

A cell production cartridge according to an embodiment includes a flow path configured to deliver liquid or gas to be used for producing a cell. In a transport path for the liquid or the gas, the flow path includes a pressure loss generating part structured so that a cross-sectional area of the liquid or the gas delivered through the flow path is smaller than cross-sectional areas before and after the pressure loss generating part.

### CROSS-REFERENCE TO RELATED APPLICATIONS

This application is based upon and claims the benefit of priority from Japanese Patent Application No. 2023-174522, filed on Oct. 6, 2023, and Japanese Patent Application No. 2024-173212, filed on Oct. 2, 2024; the entire contents of which are incorporated herein by reference.

### FIELD

Embodiments described herein relate generally to a cell production cartridge and a cell production apparatus.

### BACKGROUND

Production of induced pluripotent stem (iPS) cells includes a plurality of processes. Further, each of the processes includes a plurality of steps that use mutually-different flow paths. Conventionally known are a cell production apparatus having a closed system and configured to carry out a part of the processes or steps among the plurality of processes and a cell production apparatus configured to carry out a part of the processes or steps according to a user's operation. According to these conventional techniques, because multiple apparatuses are separately used for carrying out the processes or the steps until the production of the iPS cells is completed, it is necessary to transfer and receive cells, solutions, and the like between the multiple apparatuses. For this reason, to take a countermeasure against leakage of infectious substances to the outside and contaminations, it is necessary to use a safety cabinet accommodating all of the multiple apparatuses or a facility such as a laboratory compliant with Biosafety Level 2 (BSL-2).

A possible solution for reducing the risk of the contaminations and the like to the outside of the apparatuses is to carry out all the processes in the iPS cell production in a single closed-system apparatus; however, because required liquid delivery speeds and occurring losses of pressure (pressure losses) are mutually different among the plurality of processes, it is difficult to configure, design, and select a liquid delivery drive mechanism for controlling liquid deliveries in the processes in a single apparatus.

A cell production cartridge according to an embodiment includes a flow path configured to deliver liquid or gas to be used for producing a cell. In a liquid delivery path, the flow path includes a pressure loss generating part structured so that a cross-sectional area of the liquid or the gas delivered through the flow path is smaller than cross-sectional areas before and after the pressure loss generating part.

### BRIEF DESCRIPTION OF THE DRAWINGS

FIG. 1 is a drawing illustrating an example of a schematic configuration of a cartridge used in a cell production apparatus according to an embodiment;

FIG. 2 is a drawing illustrating an example of a schematic configuration of the cell production apparatus loaded with cartridges according to the embodiment;

FIG. 3 is a chart illustrating an example of a flow in an iPS cell production process realized by the cell production apparatus according to the embodiment;

FIG. 4 is a chart illustrating an example of a flow of steps in a blood introducing process and a blood corpuscle separating process according to the embodiment;

FIG. 5 is a diagram illustrating an example of a flow path configuration corresponding to the blood corpuscle separating process and being included in the cartridge according to the embodiment;

FIG. 6 is a drawing illustrating an exemplary configuration of a spiral flow path according to the embodiment;

FIG. 7 is a drawing for explaining a pressure loss in the flow path according to the embodiment;

FIG. 8 is a drawing for explaining liquid delivery control using gas pressure according to the embodiment;

FIG. 9 is a diagram illustrating an example of a liquid delivery drive using a pressure decrease according to the embodiment;

FIG. 10 is a diagram illustrating an example of a liquid delivery drive using a pressure increase according to the embodiment;

FIG. 11 is a diagram illustrating an example of a liquid delivery path in the blood introducing step according to the embodiment;

FIG. 12 is a diagram illustrating an example of a liquid delivery path in a blood filtering step according to the embodiment;

FIG. 13 is a diagram illustrating an example of a liquid delivery path in a first Phosphate-Buffered Saline (PBS) rinse suction step according to the embodiment;

FIG. 14 is a diagram illustrating an example of a liquid delivery path in a PBS rinse step according to the embodiment;

FIG. 15 is a diagram illustrating an example of a liquid delivery path in a second PBS rinse suction step according to the embodiment;

FIG. 16 is a diagram illustrating an example of a liquid delivery path in a cell collecting step according to the embodiment;

FIG. 17 is a diagram illustrating an example of a liquid delivery path in a first classifying step according to the embodiment;

FIG. 18 is a diagram illustrating an example of a liquid delivery path in a second classifying step according to the embodiment;

FIG. 19 is a drawing illustrating an example of a pressure loss member according to the embodiment;

FIG. 20 is a drawing illustrating an example of a state in which the pressure loss member according to the embodiment is placed in a pipe;

FIG. 21 is a drawing illustrating an example of a reduced part of a pipe according to the embodiment;

FIG. 22 is a drawing illustrating an example of an enlarged part of a pipe according to the embodiment;

FIG. 23 is a drawing illustrating an example of a pressure loss member according to a first modification example;

FIG. 24 is a drawing illustrating an example of a pressure loss member according to a second modification example;

FIG. 25 is a drawing illustrating an example of a pressure loss member according to a third modification example;

FIG. 26 is a drawing illustrating an example of a pressure loss member according to a fourth modification example;

FIG. 27 is a drawing illustrating an example of a pressure loss member according to a fifth modification example;

FIG. 28 is a drawing illustrating an example of a pressure loss member according to a sixth modification example;

FIG. 29 is a drawing illustrating an example of a state in which the pressure loss member according to the sixth modification example is placed in a pipe;

FIG. 30 is a diagram illustrating an example of an installation position of a pressure loss member according to an eighth modification example; and

FIG. 31 is a diagram illustrating another example of the installation position of the pressure loss member according to the eighth modification example.

### DETAILED DESCRIPTION

Exemplary embodiments of a cell production cartridge and a cell production apparatus will be explained in detail below, with reference to the accompanying drawings.

### EMBODIMENTS

One of the problems to be solved by the embodiments set forth in the present disclosure including the drawings is to provide an apparatus configured to carry out a plurality of liquid or gas delivery processes while differences in pressure losses among the liquid or gas delivery processes are kept within an appropriate range. It should be noted, however, that the problems to be solved by the embodiments set forth in the present disclosure including the drawings are not limited to the above problem. It is also possible to consider the problems corresponding to advantageous effects achieved by the configurations described in the following embodiments as other problems.

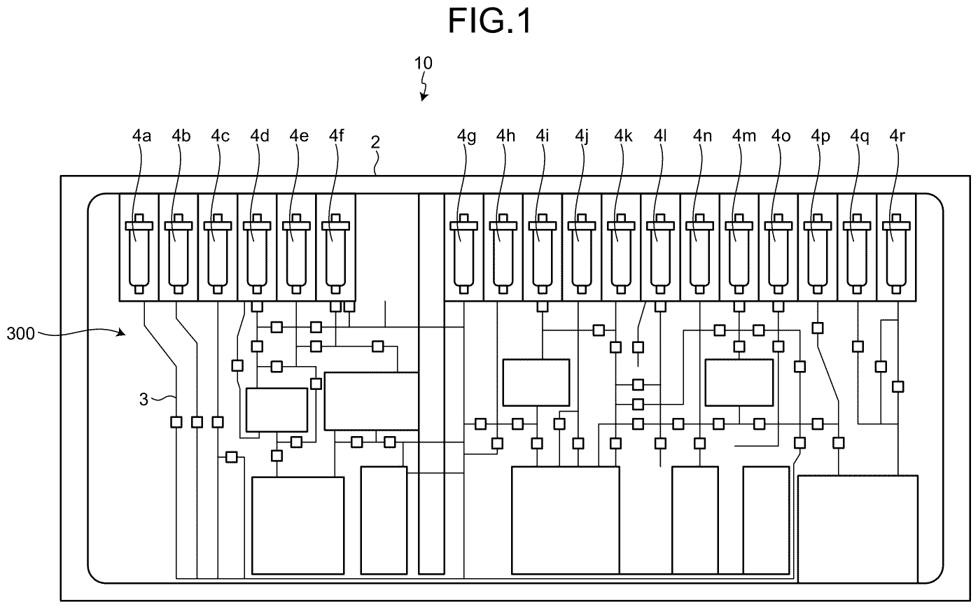

FIG. 1 is a drawing illustrating an example of a schematic configuration of a cartridge 10 used in a cell production apparatus according to an embodiment of the present disclosure. The cartridge 10 is configured to carry out all the processes throughout a cell production. In the present embodiment, it is assumed that the cartridge 10 is for producing, in particular, induced pluripotent stem (iPS) cells. The cartridge 10 is an example of a cell production cartridge of the present embodiments.

As illustrated in FIG. 1, the cartridge 10 of the present embodiment is an apparatus having a closed system and including a cover 2, pipes 3, and a plurality of containers 4a to 4r. Although the cartridge 10 of the present embodiment further includes a filter, a spiral flow path, a drive receiving part, and the like, explanations thereof will be omitted from FIG. 1.

The plurality of containers 4a to 4r store therein various types of liquids used for producing the iPS cells and liquid occurring during the production of the iPS cells. The liquids used for producing the iPS cells may be one or more specimens and reagents. Specific examples thereof include blood, a cell suspension, and physiological saline. As the physiological saline, for example, Phosphate-Buffered Saline (PBS) may be used. Further, a specimen used in the cartridge 10 may be human blood, for example. Among the plurality of containers 4a to 4r, a container storing therein a specimen used in the production of the iPS cells will be referred to as a specimen container, whereas a container storing therein a reagent will be referred to as a reagent container. Specific examples of the specimen container include a blood pack. Specific examples of the reagent container include a chemical liquid pack. The specimen does not necessarily need to be blood and may be one of other cell suspensions or the like. The liquid flowing through a flow path in the cartridge 10 of the present embodiment may be referred to as a fluid.

Among the plurality of containers 4a to 4r, the containers other than specimen containers and reagent containers such as the blood pack and the chemical liquid pack will be referred to as liquid storing containers. Specific examples of the liquid storing containers include an intermediate container and a collection container. In the following sections, when not being individually specified, the containers 4a to 4r will simply be referred to as containers 4.

For example, the plurality of pipes 3 are pipes through which various types of liquids and gases used in the production of the iPS cells flow. As the pipes 3, silicone tubes may be used, for example.

The pipes 3 and the containers 4 structure, in the cartridge 10, flow paths 300 configured to deliver any of the liquids and the gasses used in the production of the iPS cells. In the cartridge 10 of the present embodiment, the flow paths 300 are closed-system flow paths. Accordingly, the flow paths 300 in the cartridge 10 are configured to prevent the liquids and substances from leaking to the outside of the flow paths 300 and to prevent unwanted substances from inadvertently entering from the outside. Within the flow paths 300, paths through which any of the liquids and the gasses flows will be referred to as transport paths or simply lines. Among the transport paths, paths through which liquid flows may be referred to as liquid delivery paths. In contrast, among the transport paths, paths through which gas flows may be referred to as intake paths or discharge paths.

The cover 2 is configured to cover the outside of the cartridge 10. The cover 2 is configured to isolate the interior of the cartridge 10 from an external environment and to thereby prevents leakage to the outside of the cartridge 10, even in case a substance such as a liquid leaks from any of the closed-system flow paths in the cartridge 10. In other words, the cartridge 10 is provided with a double closed structure using the cover 2 and the closed-system flow paths. The double closed structure may be referred to as a double seal structure. Further, because the cartridge 10 after use is to be discarded after being sterilized, risks are reduced regarding infections that may be caused by the blood or the like used for the production of the iPS cells. Among constituent elements of a cell production apparatus 1, the cartridge 10 may be disposable and may be discarded after every session of iPS cell production, for example. In contrast, other constituent elements (e.g., a liquid delivery apparatus 11, a valve open/close apparatus 12, a cooling apparatus 13, an incubator 14, and a moving apparatus 15) may be used in the iPS cell production multiple times. Another arrangement is also acceptable in which, after the pipes 3, the containers 4, the filter, and the like in the cartridge 10 are replaced, a main body of the cartridge 10 is used multiple times. In other words, the cartridge 10 according to the present embodiment is equipment that is independently removable from the cell production apparatus (explained later) while the closed system is maintained. Further, it is possible to remove the cartridge 10 without damaging the cell production apparatus. It is assumed that, in the present embodiment, situations in which the closed system is maintained also include those in which gas flows in and out via the filter.

The quantities, the positional arrangements, and the shapes of the plurality of containers 4a to 4r and the pipes 3 illustrated in FIG. 1 are merely examples, and possible embodiments are not limited to these examples.

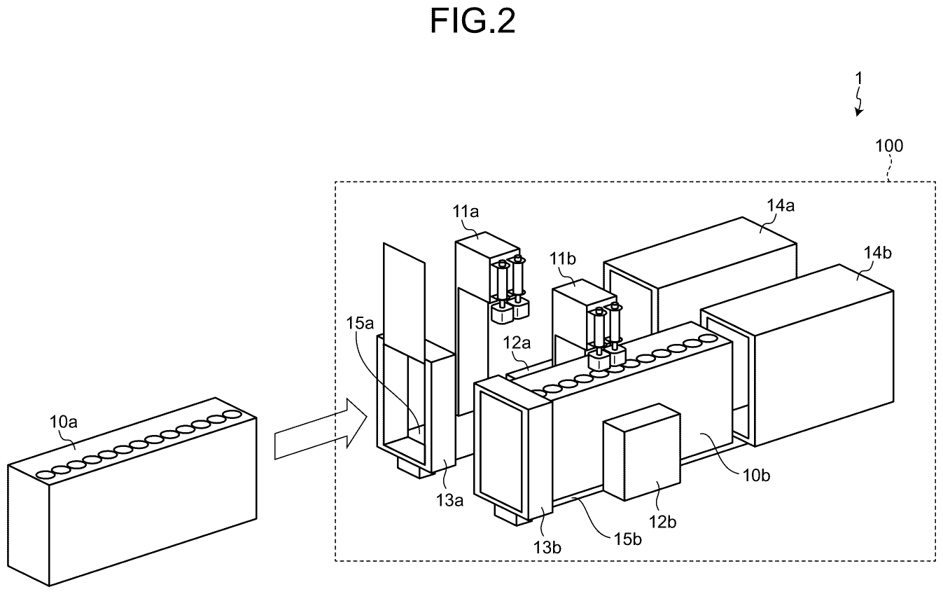

FIG. 2 is a drawing illustrating an example of a schematic configuration of the cell production apparatus 1 loaded with cartridges 10a and 10b according to the present embodiment. As illustrated in FIG. 2, the cell production apparatus 1 of the present embodiment includes the cartridges 10a and 10b, liquid delivery apparatuses 11a and 11b, valve open/close apparatuses 12a and 12b, cooling apparatuses 13a and 13b, incubators 14a and 14b, and moving apparatuses 15a and 15b. In addition, the cell production apparatus 1 includes processing circuitry configured to control the entirety of the cell production apparatus 1.

The liquid delivery apparatuses 11a and 11b are configured to drive liquid deliveries within the cartridges 10a and 10b, by applying a gas pressure increase or decrease to the liquid storing containers of the cartridges 10a and 10b. The liquid delivery apparatuses 11a and 11b are provided, for example, with a regulator configured to control intensities of the gas pressure to be increased or decreased, a pressure pad configured to transmit the pressure to the cartridges 10a and 10b, and a moving mechanism configured to move the pressure pad to a liquid storing container to which the pressure increase or decrease is to be applied. Further, the liquid delivery apparatuses 11a and 11b may further be provided with a computer or the like for controlling the liquid deliveries.

The valve open/close apparatuses 12a and 12b are configured to change the liquid delivery paths within the cartridges 10a and 10b, by opening and closing valves provided for the pipes in the cartridges 10a and 10b.

The cooling apparatuses 13a and 13b are configured to cool one or more specimens and reagents filling the cartridges 10a and 10b in advance, to appropriate temperatures. It is possible to open and close the cooling apparatuses 13a and 13b, so that an operator can insert the cartridges 10a and 10b into a main body part 100 of the cell production apparatus 1 through opening parts of the cooling apparatuses 13a and 13b. Before being inserted into the cell production apparatus 1, the cartridges 10 are filled with the one or more specimens and reagents by the operator in advance.

The incubators 14a and 14b are configured to maintain an environment suitable for culturing cells, by keeping temperature, humidity, a carbon dioxide concentration and the like in the cartridges 10a and 10b constant. At the time of culturing the cells, the cartridges 10a and 10b are inserted by the moving apparatuses 15a and 15b into the incubators 14a and 14b.

The moving apparatuses 15a and 15b are configured to move positions of the cartridges 10a and 10b within the main body part 100 of the cell production apparatus 1.

In the example illustrated in FIG. 2, the single cell production apparatus 1 includes two each of the following: the cartridges 10a and 10b; the liquid delivery apparatuses 11a and 11b; the valve open/close apparatuses 12a and 12b; the cooling apparatuses 13a and 13b; the incubators 14a and 14b; and the moving apparatuses 15a and 15b; however, this configuration is merely an example. The cell production apparatus 1 may include one each of these apparatuses or may include three or more each of these apparatuses. In the following sections, when not being distinguished from each other, the two of the cartridges 10a and 10b, the liquid delivery apparatuses 11a and 11b, the valve open/close apparatuses 12a and 12b, the cooling apparatuses 13a and 13b, the incubators 14a and 14b, and the moving apparatuses 15a and 15b will simply be referred to as cartridges 10, liquid delivery apparatuses 11, valve open/close apparatuses 12, cooling apparatuses 13, incubators 14, and moving apparatuses 15. In addition, the cell production apparatus 1 may further include a control apparatus and the like that are not illustrated in FIG. 2. It is assumed that the cell production apparatus 1 according to the present embodiment includes, at least, a cartridge 10 and a liquid delivery apparatus 11.

Next, an iPS cell production process performed by the cell production apparatus 1 will be explained. An iPS cell is a cell that is produced by introducing a specific gene into a somatic cell and subsequently culturing the cell and has a capability of being differentiated into a cell of various tissues and organs. In the present embodiment, it is assumed that, as a material of the iPS cells, nucleated cells in somatic cells, in particular, white blood corpuscles are to be used.

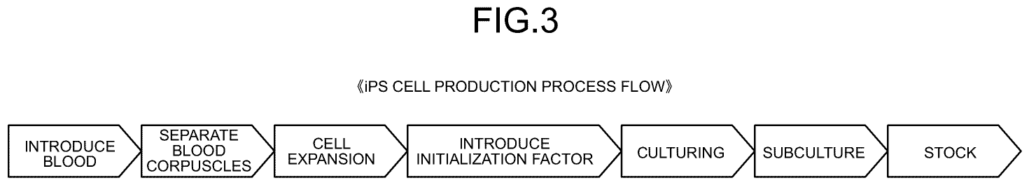

FIG. 3 is a chart illustrating an example of a flow in an iPS cell production process realized by the cell production apparatus 1 according to the present embodiment. As illustrated in FIG. 3, the iPS cell production includes a blood introducing process, a blood corpuscle separating process, a cell expansion process, an initialization factor introducing process, a culturing process, a subculture process, and a stock process.

In the present embodiment, each of the individual processes related to the cell production illustrated in FIG. 3 will be referred to as a “process”. Further, each of the processes includes one or more single actions of causing liquid such as blood or physiological saline to flow. In the present embodiment, each of the individual liquid delivery actions included in the processes will be referred to as a “step” or a “liquid delivery process”.

Although all the processes illustrated in FIG. 3 are performed by the cell production apparatus 1, in the present embodiment, in particular, the blood introducing process and the blood corpuscle separating process will be explained as examples.

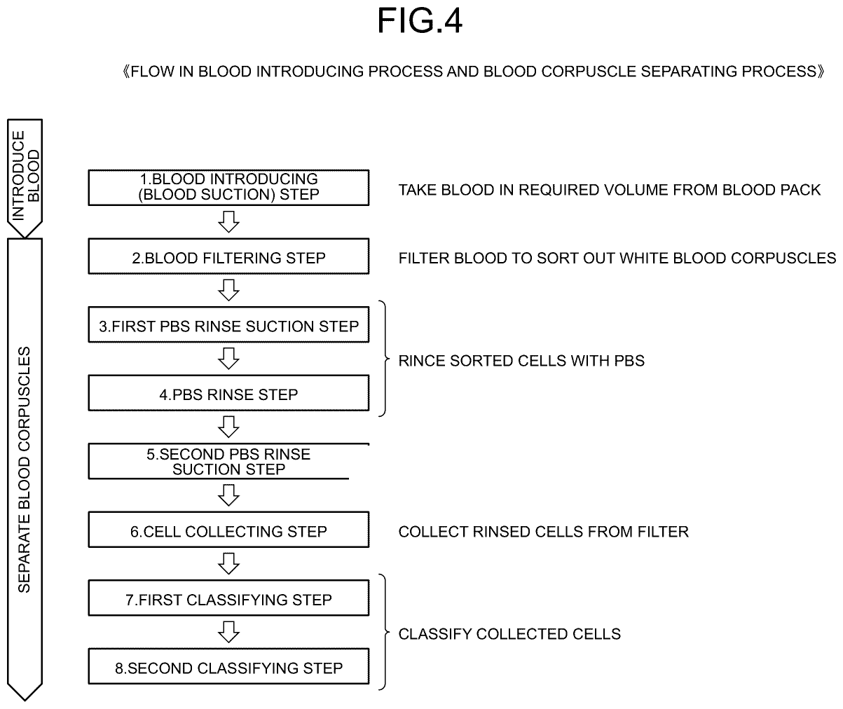

FIG. 4 is a chart illustrating an example of a flow of steps in the blood introducing process and the blood corpuscle separating process according to the embodiment. As illustrated in FIG. 4, the blood introducing process includes a blood introducing (blood suction) step. Further, the blood corpuscle separating process in an example includes seven steps, namely, a blood filtering step, a first PBS rinse suction step, a PBS rinse step, a second PBS rinse suction step, a cell collecting step, a first classifying step, and a second classifying step. In the present example, the eight steps included in the blood introducing process and the blood corpuscle separating process will be described as a series of actions in a flow.

The first step called the blood introducing step is a liquid delivery action in which blood in a required amount is introduced into the pipes 3 in the flow paths 300. In other words, the blood introducing step is the liquid delivery action to bring the blood in the required amount into the pipes 3 in the flow paths 300 from the blood pack and may therefore be referred to as a blood suction step.

The blood corpuscle separating process is performed for the purpose of sorting out white blood corpuscles from the blood and further taking out only specific cells therefrom. The specific cells may be, for example, certain white blood corpuscles in which the nucleus inside is not broken.

The second step called the blood filtering step is a liquid delivery action to filter the blood for the purpose of sorting out the white blood corpuscles from the blood. In this situation, because granularity of the sorting in the blood filtering step is coarser than granularity of the first and the second classifying steps at later stages, the sorted result at this stage also includes other blood cells in addition to the specific cells being desired.

The third step called the first PBS rinse suction step and the fourth step called the PBS rinse step are liquid delivery actions to rinse the cells sorted by the filter, while using PBS.

The fifth step called the second PBS rinse suction step is a liquid delivery action to suck the PBS as a pre-processing process for the upcoming cell collecting step.

The sixth step called the cell collecting step is a liquid delivery action to collect the rinsed cells from the filter.

The seventh step called the first classifying step and the eighth step called the second classifying step are liquid delivery actions to classify the cells collected from the filter. As a result, the desired specific cells are extracted.

Next, the flow paths 300 for realizing the steps illustrated in FIG. 4 will be explained.

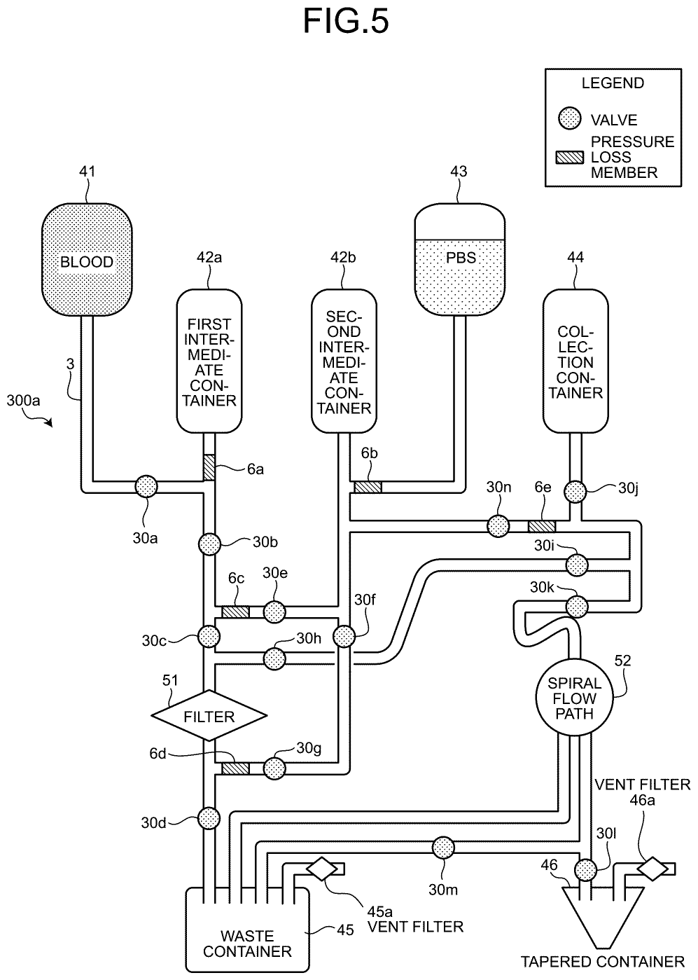

FIG. 5 is a diagram illustrating an example of a flow path configuration corresponding to the blood corpuscle separating process and being included in the cartridge 10 according to the embodiment. A flow path 300a illustrated in FIG. 5 is a part of the flow paths 300 included in the cartridge 10.

As a premise, the liquids delivered to the flow path 300a are, as presented in Table 1 below, a chemical liquid, such as PBS, and blood. The density of each of the liquids delivered to the flow path 300a is approximately equal to that of water. The viscosity levels of the liquids range from a substantially equal level to water to approximately five times higher. In addition, a process request regarding flow rates at the time of delivering the liquids to the flow path 300a is in the range of 0.1 mL/min to 50 mL/min.

TABLE 1

| | Chemical liquid (e.g., | |
|---|---|---|
| Liquid | PBS) | Blood |
| Density | 1000 | 1000 |
| [kg/m³] | (Equal to water) | (Equal to water) |
| Viscosity | 0.00093 | Approximately 5 times |
| [Pa · s] | (Equal to water at 23° C.) | higher than water |

As illustrated in FIG. 5, the flow path 300a includes the plurality of pipes 3, valves 30a to 30n, pressure loss devices 6a to 6e, a filter 51, a spiral flow path 52, a blood pack 41, a first intermediate container 42a, a second intermediate container 42b, a chemical liquid pack 43, a collection container 44, a waste container 45, and a tapered container 46.

The blood pack 41, the first intermediate container 42a, the second intermediate container 42b, the chemical liquid pack 43, and the collection container 44 are examples of the containers 4 included in the cartridge 10. Further, among these, the first intermediate container 42a, the second intermediate container 42b, and the collection container 44 are examples of the liquid storing containers. As a result of a pressure increase or a pressure decrease of the gas pressure being applied from the liquid delivery apparatus 11 to the first intermediate container 42a, the second intermediate container 42b, and the collection container 44, the liquid stored therein is delivered. Details of liquid delivery control using the gas pressure will be explained later.

Further, in the present embodiment, the path in which the liquid flows through the flow path 300a due to the liquid delivery action in each of the steps will be referred to as a liquid delivery path. The liquid delivery path is different for each of the steps.

The blood pack 41 contains the blood to be used as a material of the iPS cells. While containing the blood, the blood pack 41 is attached to the cartridge 10 by an operator.

In the first intermediate container 42a and the second intermediate container 42b, either the blood or the chemical liquid such as PBS is temporarily stored in intermediate parts of the flow path 300a.

The chemical liquid pack 43 is attached to the cartridge 10 by the operator while containing the chemical liquid such as physiological saline to be used for the production of the iPS cells. In the present embodiment, the chemical liquid pack 43 contains PBS.

The collection container 44 is configured to store therein the cells collected from the filter 51.

The waste container 45 is configured to store therein the blood cells other than the specific cells classified from the cells collected from the filter 51.

At the end of the blood corpuscle separating process, the tapered container 46 is configured to store therein the specific cells that have been classified. The cells stored in the tapered container 46 are to be used in the cell expansion process that follows.

The pipes 3 are pipes that each have a hole through which liquid or gas flows and that connect together the filter 51, the spiral flow path 52, the blood pack 41, the first intermediate container 42a, the second intermediate container 42b, the chemical liquid pack 43, the collection container 44, the waste container 45, and the tapered container 46. It is desirable to provide the waste container 45 with a vent filter 45a capable of venting the interior of the container. The vent filter 45a also has a function of preventing bacteria from inadvertently entering the flow path. It is desirable to provide the tapered container 46 with a vent filter 46a capable of venting the interior of the container. The vent filter 46a also has a function of preventing bacteria from inadvertently entering the flow path. However, when the waste container 45 and the tapered container 46 are not each a rigid body, but have structures that are contractable and expandable, the vent filters 45a and 46a are not necessarily required.

As the pipes 3, for example, it is possible to use silicone tubes, as mentioned above. In the present embodiment, as for the size thereof, the pipes 3 may each have, for example, an outside diameter of 3 mm and an inside diameter of 1 mm, approximately. Further, in connection parts between the pipes 3, T-shaped joints, for example, may be used to realize the connections.

The valves 30a to 30n are provided for the pipes 3 and are configured to change the liquid delivery paths in the flow path 300a by opening and closing the flow paths under control of the valve open/close apparatus 12.

The pressure loss members 6a to 6e are members configured to increase pressure losses (losses of pressure) in the flow path 300a. More specifically, the pressure loss members 6a to 6e are tubues each having a through hole and are inserted in the pipes 3. Accordingly, the pressure loss members 6a to 6e may be referred to as pressure loss tubes. In the pressure loss members 6a to 6e, the holes through which liquid or gas flows are configured to be smaller than those in the pipes 3 positioned before and after the pressure loss members 6a to 6e. For this reason, for example, when the liquid flows through the flow path 300a, in a certain range of the flow path 300a that is filled with the liquid, cross-sectional areas of the liquid in each of the pressure loss members 6a to 6e are smaller than cross-sectional areas of the liquid in the pipes 3. The certain range of the flow path 300a that is filled with the liquid denotes a range corresponding to the liquid delivery path in the flow path 300a. Accordingly, in other words, the flow path 300a includes the pressure loss members 6a to 6e structured so that, in the liquid delivery path or the transport path, the cross-sectional areas of the liquid or the gas delivered through the flow path 300a are smaller than the cross-sectional areas before and after the pressure loss members 6a to 6e.

Further, as for cross-sectional areas of the holes through which the liquid flows in the flow path 300a also, cross-sectional areas of the parts having the pressure loss members 6a to 6e are smaller than cross-sectional areas of the parts structured only with the pipes 3. Accordingly, in other words, the flow paths 300 include the pressure loss members 6a to 6e structured so that, in the liquid delivery path, cross-sectional areas are smaller than cross-sectional areas before and after the pressure loss members 6a to 6e. In the following sections, when not being particularly distinguished individually, the pressure loss members 6a to 6e will simply be referred to as pressure loss members 6. The pressure loss members 6 are examples of a pressure loss generating part of the present embodiment. Further, the inside diameters of the pressure loss members 6 may serve as an example of a pressure loss generating part. Details of configurations of the pressure loss members 6 will be explained later.

The filter 51 is configured to sort out objects from the liquid flowing through the flow path 300a. More specifically, the filter 51 is configured to sort out the white blood corpuscles from the blood.

The spiral flow path 52 is an example of equipment configured to sort out objects in the liquid by using a flowing action. More specifically, the spiral flow path 52 is configured to classify, from liquid containing the cells collected from the filter 51, the desired specific cells and other blood cells. The spiral flow path 52 may also be referred to as a swirling flow path.

FIG. 6 is a drawing illustrating an exemplary configuration of the spiral flow path 52 according to the present embodiment. The spiral flow path 52 includes a micro flow path 521 spirally formed and is configured to classify particles contained in a fluid. More specifically, the plurality of particles contained in the fluid are separated according to sizes (the particle diameters) of the particles.

The micro flow path 521 is the flow path obtained by spirally forming a pipe of which the inside diameter is smaller than that of each of the pipes 3.

As a result of the liquid that flowed in through a fluid supply part 522 being delivered through the spirally-formed micro flow path 521, the blood cells in the liquid are separated so that relatively largeer cells 90 such as white blood corpuscles move toward the wall surface of the flow path and relatively smaller cells 91 such as platelets and red blood corpuscles move toward the center of the flow path, due to lift caused by a velocity distribution near the wall surface of the flow path in the micro flow path 521. Further, due to an eddy current caused by curves of the micro flow path 521, the larger cells 90 near the wall surface of the flow path move inward, whereas the smaller cells 91 at the flow path center move outward. Due to this action, at a branch part 523, the larger cells 90 flow into an inner flow path 521a, whereas the smaller cells 91 flow into an outer flow path 521b, separately from each other. As a result, the spiral flow path 52 is able to classify the blood cells in the liquid.

The classified larger cells 90, i.e., the specific cells among the white blood corpuscles, flow into a collecting part 525. In contrast, the classified smaller cells 91 flow into a discarding part 524. Although not illustrated in FIG. 6, the collecting part 525 is connected to a pipe 3 directed toward the tapered container 46 illustrated in FIG. 5. Further, the discarding part 524 is connected to a pipe 3 directed toward the waste container 45 illustrated in FIG. 5.

Alternatively, in place of the spiral flow path 52, the flow path 300a may include equipment configured to sort out the objects in the liquid by using other means.

Next, the liquid delivery control using the gas pressure according to the present embodiment will be explained. In the cell production apparatus 1 according to the present embodiment, the liquid delivery apparatus 11 is configured to cause the liquid such as the blood or the chemical liquid to flow so that each of the liquid delivery paths structuring the processes from the blood to the iPS cells has a determined flow rate. An anergy source to cause the liquid to flow may be the gas pressure (air pressure). The gas pressure may be positive pressure or negative pressure. The liquid delivery apparatus 11 is capable of delivering the liquid in an appropriate amount at an appropriate speed, by applying a pressure difference corresponding to a pressure loss determined by the configuration of the liquid delivery path as well as the viscosity, the density, and a flow rate of the flowing liquid, to a position between the upstream side and the downstream side, while taking head pressure into account. The viscosity and the density of the liquid flowing through the flow path 300a are assumed to be those presented in Table 1.

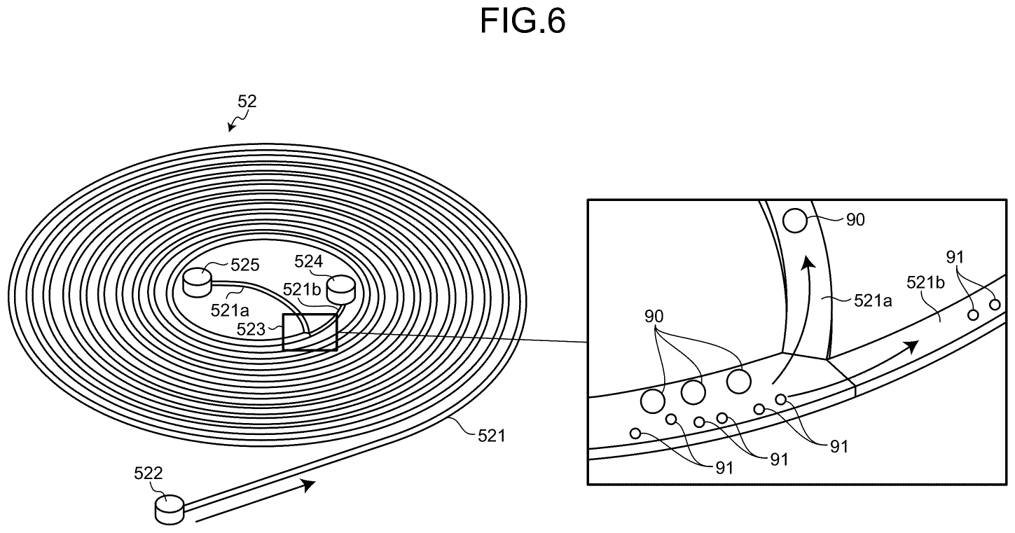

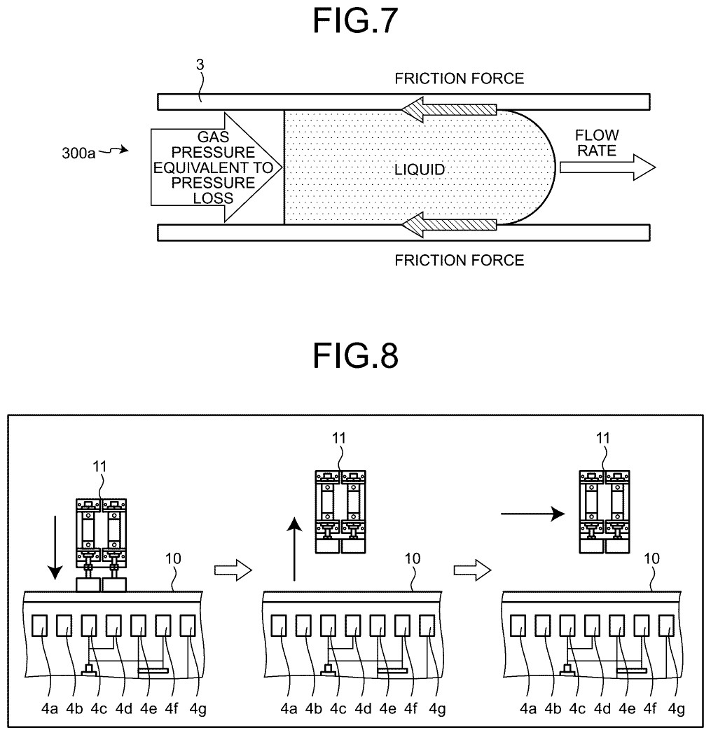

FIG. 7 is a drawing for explaining a pressure loss in the flow path according to the present embodiment. In the cell production apparatus 1 according to the present embodiment, the liquid delivery apparatus 11 is configured to control the liquid deliveries in the flow path 300a, by increasing or decreasing the gas pressure applied to the liquid storing containers. For example, as a result of the liquid delivery apparatus 11 pressing the liquid to be delivered by applying, to the liquid, gas pressure having a pressure level corresponding to the pressure loss, the liquid is caused to flow through the pipe 3. In this situation, the pressure loss is energy which the liquid loses due to friction force caused between the liquid and the inner wall of the flow path 300a. Depending on a relationship between the gas pressure and the pressure loss, the speed and the flow rate of the liquid flow change. The smaller the pressure loss in the flow path 300a is, the smaller is the gas pressure required by the liquid delivery.

FIG. 8 is a drawing for explaining the liquid delivery control using the gas pressure according to the present embodiment. In FIG. 8, for the sake of convenience in the explanation, it is assumed that containers 4a to 4g are liquid storing containers. The liquid delivery apparatus 11 is configured to change the liquid storing container that will have a pressure increase or a pressure decrease, while moving over the cartridge 10 along the direction in which the liquid storing containers are arranged in the cartridge 10. For example, the liquid delivery apparatus 11 is configured to align the position thereof with the storage position of the container 4c in the cartridge 10, to descend toward the cartridge 10, to connect to the cartridge 10, and to transmit a liquid delivery drive. After that, the liquid delivery apparatus 11 is configured to ascend away from the cartridge 10 and to subsequently move to the storage position of the next container 4d. After moving to a position above the storage position of the container 4d, the liquid delivery apparatus 11 is configured to descend toward the cartridge 10. After the descent, the liquid delivery apparatus 11 is configured to connect to the cartridge 10 and to transmit a liquid delivery drive.

As explained above, by repeatedly carrying out the moving and the pressure increase or the pressure decrease, the liquid delivery apparatus 11 is configured to control the liquid deliveries in the flow paths 300 in the cartridge 10. For example, the liquid delivery apparatus 11 according to the present embodiment includes a generic regulator, so that the magnitude of the gas pressure to be increased or decreased is controlled by the regulator. In the present example, transmitting the liquid delivery drive denotes the liquid delivery apparatus 11 causing the liquid in the flow path 300a to flow by increasing or decreasing the gas pressure. The gas pressure is motive power for the liquid deliveries. The liquid delivery apparatus 11 is configured to transmit the motive power to the liquid in the flow path 300a. In other words, the liquid delivery apparatus 11 is configured to control the liquid deliveries in the flow path 300a, by increasing or decreasing the gas pressure. Further, the liquid delivery apparatus 11 increasing or decreasing the gas pressure means, in other words, that the liquid delivery apparatus 11 increases or decreases pressure on the liquid in the flow path 300a.

The liquid delivery apparatus 11 is configured to transmit the liquid delivery drive, from the outside of the double seal structure of the cartridge 10. In other words, the liquid delivery apparatus 11 is configured to control the liquid deliveries by using the gas pressure, from the outside of the flow path 300a. Next, a configuration of the drive receiving part configured, on the cartridge 10 side, to receive the motive power for the liquid deliveries from the liquid delivery apparatus 11 will be explained.

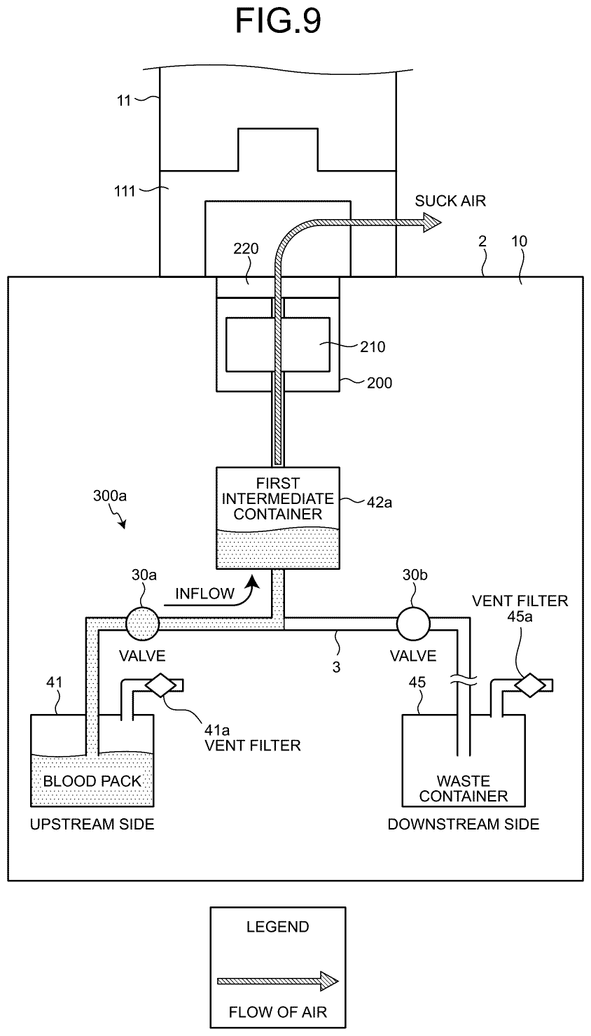

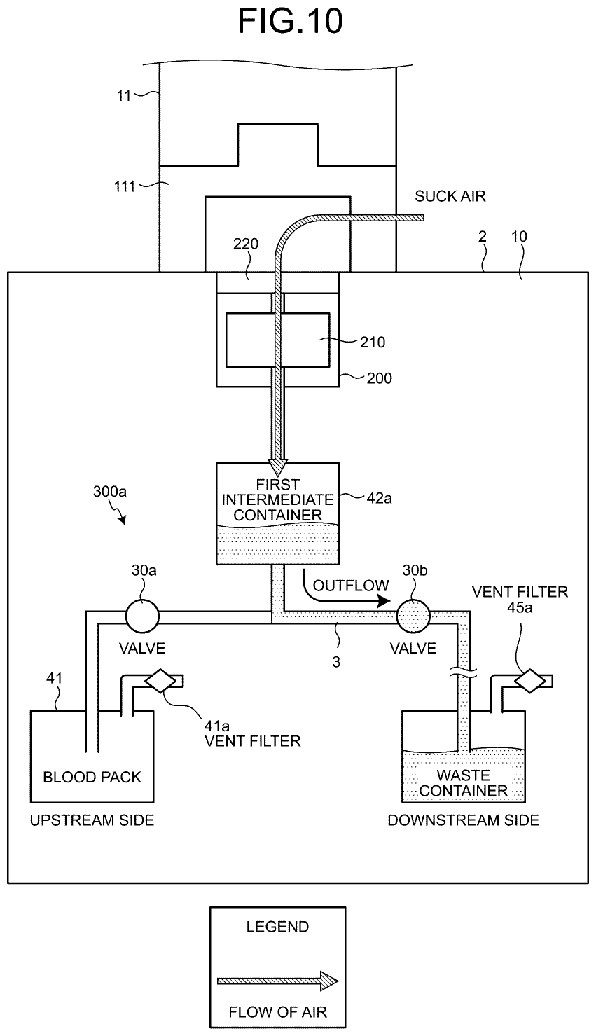

FIG. 9 is a diagram illustrating an example of the liquid delivery drive using a pressure decrease according to the present embodiment. FIG. 10 is a diagram illustrating an example of the liquid delivery drive using a pressure increase according to the present embodiment. Among the elements included in the flow path 300a illustrated in FIG. 5, for the sake of convenience in the explanation, FIGS. 9 and 10 illustrate only the blood pack 41, the first intermediate container 42a, the waste container 45, the valves 30a and 30b, and the pipes 3 connecting these elements together, while the other elements are omitted from the illustration.

In the cartridge 10, above the liquid storing containers, pressure chambers 200 are provided. FIGS. 9 and 10 illustrate, as an example, a pressure chamber 200 provided above the first intermediate container 42a. Although not illustrated in FIGS. 9 and 10, in the flow path 300a, pressure chambers 200 are provided above the second intermediate container 42b and the collection container 44, in addition to the first intermediate container 42a. In the flow path 300a, because of not being subject to the liquid delivery drive from the liquid delivery apparatus 11, the blood pack 41, the chemical liquid pack 43, the waste container 45, and the tapered container 46 are not connected to the pressure chambers 200. It is desirable to provide the blood pack 41 with a vent filter 41a capable of venting the interior of the blood pack 41.

Each of the pressure chambers 200 includes a disc filter 210 and a sheet filter 220. The sheet filter 220 is provided in an open part of the cover 2 of the cartridge 10 and gas (air) is sucked therefrom and applied thereto by the liquid delivery apparatus 11. Further, the liquid delivery apparatus 11 bringing the pressure chamber 200 into an open state without sucking or applying the gas is referred to as venting. When being vented, because the gas in the pressure chamber 200 is released to the outside, the pressure inside the pressure chamber 200 is decreased. The disc filter 210 is provided more inward within the pressure chamber 200 relative to the sheet filter 220. The sheet filter 220 and the disc filter 210 let gas pass, but do not pass unwanted substances from the outside of the cartridge 10. The path for the gas is protected from external contaminations by a double structure using the sheet filter 220 and the disc filter 210. The pressure chamber 200 is an example of the drive receiving part of the present embodiment. The pressure chamber 200 is configured to receive the liquid delivery drive using the gas pressure, from the liquid delivery apparatus 11. In other words, the pressure chamber 200 is configured to receive the liquid delivery control on the liquid exercised by the liquid delivery apparatus 11. That is to say, the pressure chamber 200 is configured to receive the motive power for the liquid deliveries, from the liquid delivery apparatus 11 provided external to the cartridge 10. Further, more specifically, by receiving, from the liquid delivery apparatus 11, the pressure increase or the pressure decrease of the gas pressure serving as the motive power for the liquid deliveries, the pressure chamber 200 is configured to receive the transmission of the motive power for the liquid. Further, not only the pressure chambers 200, but also the flow paths 300 and 300a may be considered as examples of the drive receiving part.

A pressure pad 111 of the liquid delivery apparatus 11 is connected as being aligned with the section of the cartridge 10 having the sheet filter 220, so that the gas is sucked and applied through the sheet filter 220 and the disc filter 210. The pressure pad 111 may be referred to as a pressurizing pad.

Normally, the flow of the liquid in the flow path 300a under the liquid delivery drive travels to an upstream-side container, a liquid storing container, and a downstream-side container in the stated order. In the examples in FIGS. 9 and 10, the first intermediate container 42a serving as the liquid storing container is in the middle, while the blood pack 41 serves as the upstream-side container, whereas the waste container 45 serves as the downstream-side container.

As illustrated in FIG. 9, when the pressure pad 111 of the liquid delivery apparatus 11 sucks the gas, the pressure of the pressure chamber 200 is decreased. The pressure chamber 200 is configured to receive the drive on the blood realized by the pressure decrease. In that situation, the blood flows from the blood back 41 on the upstream side into the first intermediate container 42a. With contraction of the blood pack 41, it is possible to cause the blood to flow from the blood pack 41 into the first intermediate container 42a. The liquid delivery apparatus 11 is configured to control the magnitude of the gas pressure by controlling a suction amount of the gas, so that the blood in an amount required by the production of the iPS cells is introduced to the first intermediate container 42a. In this situation, the valve open/close apparatus 12 is configured to open the valve 30a and to close the valve 30b, so that no blood flows toward the waste container 45.

Further, as illustrated in FIG. 10, when the pressure pad 111 of the liquid delivery apparatus 11 applies the gas, the pressure of the pressure chamber 200 is increased. The pressure chamber 200 is configured to receive the drive on the blood realized by the pressure increase. In that situation, the blood flows out from the first intermediate container 42a to the waste container 45 on the downstream side. The liquid delivery apparatus 11 is configured to control the speed of the outflowing blood, by controlling the amount of the applied gas and thereby controlling the magnitude of the gas pressure. In this situation, the valve open/close apparatus 12 is configured to open the valve 30b and to close the valve 30a, so that no blood flows toward the blood pack 41.

As explained above, the cell production apparatus 1 according to the present embodiment is configured to control the liquid deliveries in the flow path 300a by increasing or decreasing the gas pressure to the liquid storing containers and is therefore provided with no liquid delivery drive mechanism such as an actuator in intermediate parts of the liquid delivery paths. For this reason, even when there is a difference in the pressure losses within the liquid delivery path, it would be difficult to fine-tune the intensity of the liquid delivery drive for each location. In addition, when the pressure loss of the entire liquid delivery path is extremely small, because the degree of difficulty for controlling the gas pressure becomes higher, it would be difficult to configure, design, or select a liquid drive mechanism.

In the present embodiment, the single apparatus (the liquid delivery apparatus 11) is configured to control the gas pressure for the liquid deliveries in all the liquid or gas delivery processes. For this reason, when there is a large difference in total pressure losses of the liquid delivery paths among the liquid or gas delivery processes, because the magnitude of the gas pressure required at the time of the liquid delivery varies greatly, the degree of difficulty of the liquid delivery control required of the liquid delivery apparatus 11 becomes higher. In particular, when settings of the liquid delivery apparatus 11 are configured in accordance with a liquid delivery path that inevitably has a large pressure loss such as the liquid delivery path including the spiral flow path 52, it would be difficult to control the gas pressure in other liquid delivery paths having a smaller total pressure loss.

To cope with the circumstances described above, for the flow path 300a having a plurality of liquid delivery paths like in the cartridge 10 according to the present embodiment, it is possible to facilitate the control on the liquid delivery drive, by using pressure loss members 6 so that differences in the pressure losses among the liquid delivery paths are kept within an appropriate range.

It is desirable to ensure that a total pressure loss of each of the liquid delivery paths included in the flow paths 300 falls in a range where the difference from a total pressure loss of a liquid delivery path having the largest pressure loss is not too large, while the liquid delivery path having the largest pressure loss within the flow paths 300 is used as a reference. More specifically, it is desirable to ensure that the total pressure loss of each of the liquid delivery paths is equal to or smaller than the total pressure loss of the liquid delivery path having the largest pressure loss, while being kept within a range that is easily controlled by a regulator configured to be able to control the total pressure loss of the liquid delivery path having the largest pressure loss. When each of the liquid delivery paths satisfies this condition, it becomes easy to select and to establish settings of a regulator product for the liquid delivery apparatus 11.

In the present embodiment, among all the liquid delivery paths in the flow paths 300 included in the cartridge 10, the liquid delivery path having the largest pressure loss is the liquid delivery path including the spiral flow path 52. A total pressure loss of the liquid delivery path including the spiral flow path 52 is, for example, approximately in the range of 250 kPa to 260 kPa, inclusive. Among generic regulators, when a regulator capable of controlling the gas pressure in the range of 250 kPa to 260 kPa is used for controlling the liquid deliveries in the liquid delivery paths in the flow path 300a, it is desirable to ensure that, with respect to each of the liquid delivery paths, a total pressure loss of the entire liquid delivery path is kept within the range of 20 kPa to 260 kPa. Further, considering a stabilization zone for stable operations of the regulator, it is especially desirable to keep the total pressure loss of the entire liquid delivery path within the range of 30 kPa to 260 kPa. However, the value of the total pressure loss is merely an example and may fluctuate depending on configurations of the flow paths 300 and other conditions.

In the cartridge 10 according to the present embodiment, the pressure loss members 6 are provided in the flow path 300a, for the purpose of ensuring, with respect to certain liquid delivery paths that are among the liquid delivery paths included in the flow path 300a and have a smaller pressure loss than the desirable value range, that the pressure loss of the entire liquid delivery path is increased so as to fall within the desirable value range.

Next, the liquid delivery path in each of the steps in the flow path 300a according to the present embodiment will be explained. Table 2 presented below is a table indicating the liquid delivery control using the gas pressure, in each of the eight steps included in the blood introducing process and the blood corpuscle separating process.

TABLE 2

| | | First | Second | |
|---|---|---|---|---|
| | | intermediate | intermediate | Collection |
| Step | Liquid delivery details | container | container | container |
| 1 | Blood suction | Decrease | | |
| | | pressure | | |
| 2 | Blood filtering | Increase | | |
| | | pressure | | |
| 3 | First PBS rinse suction | | Decrease | |
| | | | pressure | |
| 4 | PBS rinse | | Increase | |
| | | | pressure | |
| 5 | Second PBS rinse suction | | Decrease | |
| | | | pressure | |
| 6 | Cell collection | | Increase | Vented |
| | | | pressure | |
| 7 | First classification | | | Increase |
| | (Flows into waste | | | pressure |
| | container until | | | |
| | classification | | | |
| | stabilizes) | | | |
| 8 | Second classification | | | Increase |
| | (Flows into tapered | | | pressure |
| | container after | | | |
| | classification stabilizes) | | | |

As presented in Table 2, as a result of the pressure pad 111 of the liquid delivery apparatus 11 exercises the pressure control while moving to the first intermediate container 42a, the second intermediate container 42b, and the collection container 44 in the stated order, the blood and the chemical liquid are delivered through the flow path 300a.

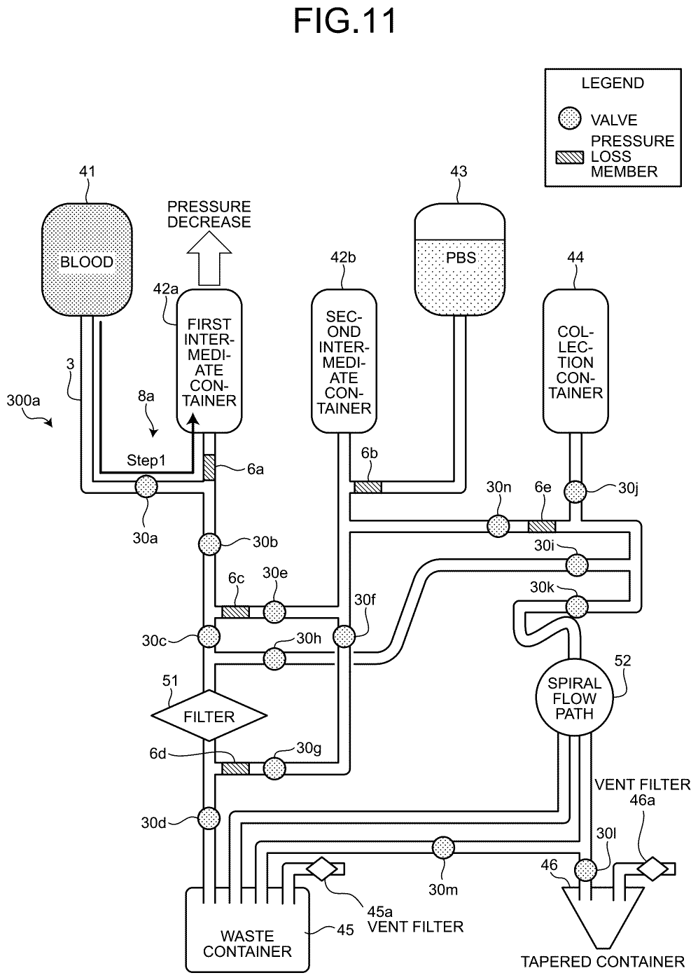

Next, the liquid delivery paths in each of the eight steps will be explained, with reference to FIGS. 11 to 18. In FIGS. 11 to 18, although the opening and the closing of the valves 30a to 30n will be omitted from the explanations, the valve open/close apparatus 12 is configured to close certain valves 30 that are outside the liquid delivery paths and to open certain valves 30 that are inside the liquid delivery paths.

FIG. 11 is a diagram illustrating an example of a liquid delivery path 8a in the blood introducing step according to the present embodiment. As a result of the liquid delivery apparatus 11 decreasing the pressure of the first intermediate container 42a, the blood is delivered from the blood pack 41 to the first intermediate container 42a. Although the blood pack 41 is not under the gas pressure control exercised by the liquid delivery apparatus 11, as a result of the liquid delivery apparatus 11 decreasing the pressure of the first intermediate container 42a provided on the inflow side in this manner, it is possible to exercise the liquid delivery control over the blood stored in the blood pack 41.

The location where the blood flows from the blood pack 41 to the first intermediate container 42a corresponds to the liquid delivery path 8a in the blood introducing step. The liquid delivery path 8a includes the pressure loss member 6a provided before the first intermediate container 42a.

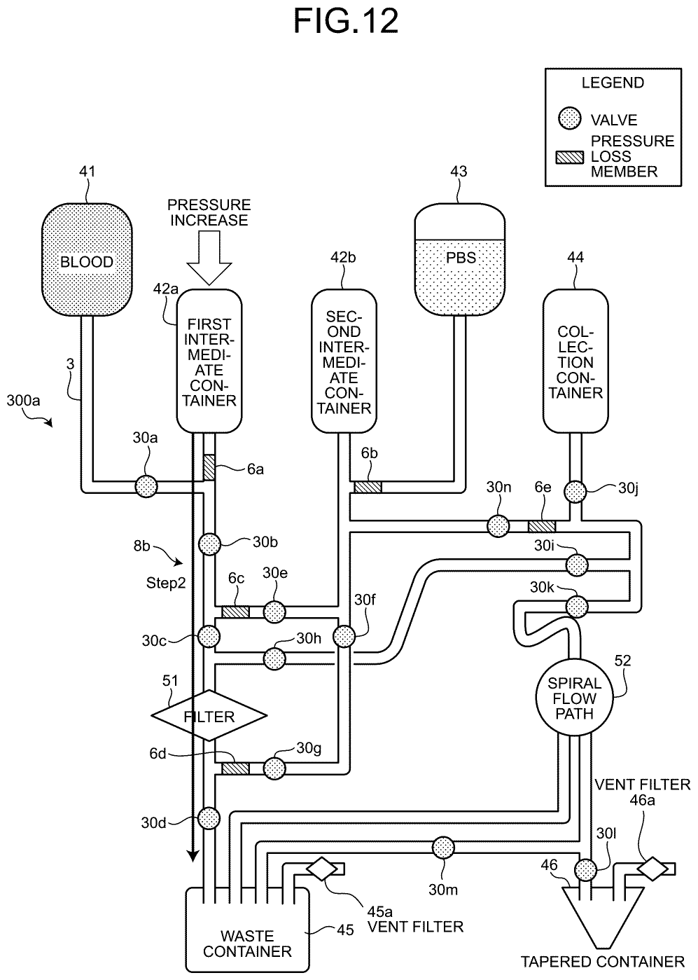

FIG. 12 is a diagram illustrating an example of a liquid delivery path 8b in the blood filtering step according to the present embodiment. As a result of the liquid delivery apparatus 11 increasing the pressure of the first intermediate container 42a, the blood is delivered from the first intermediate container 42a, via the filter 51, to the waste container 45. The location where the blood flows from the first intermediate container 42a to the waste container 45 corresponds to the liquid delivery path 8b in the blood filtering step. The liquid delivery path 8b includes the filter 51.

To properly filter the white blood corpuscles, it is important to control the speed of the blood flowing into the filter 51. When the speed of the blood inflow into the filter 51 is higher than an appropriate level, the white blood corpuscles may be broken by the pressure, or the sorting may be inefficient because the blood does not spread over the entire filter 51. For this reason, the liquid delivery path 8b in the flow path 300a includes the pressure loss member 6a provided on the upstream side of the filter 51. By the pressure loss member 6a, the speed of the blood flowing into the filter 51 is decreased.

Further, because the pressure loss member 6a is provided in a straight part of the pipe 3 connected to the first intermediate container 42a, the liquid delivery path 8a illustrated in FIG. 11 and the liquid delivery path 8b illustrated in FIG. 12 are able to share the single pressure loss member 6a.

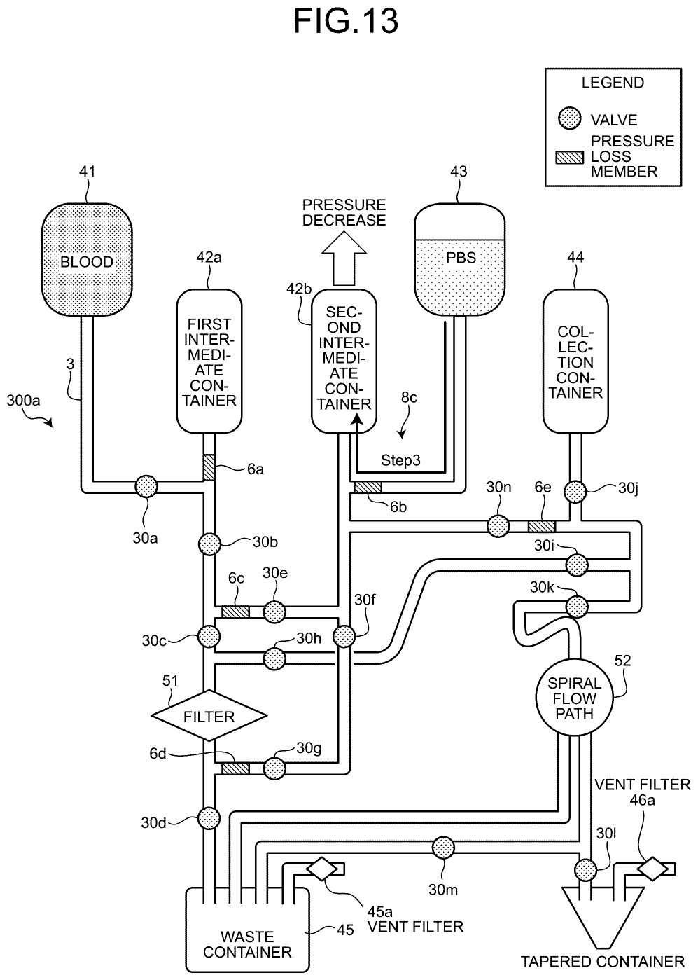

FIG. 13 is a diagram illustrating an example of a liquid delivery path 8c in the first PBS rinse suction step according to the present embodiment. As a result of the liquid delivery apparatus 11 decreasing the pressure of the second intermediate container 42b, the PBS is delivered from the chemical liquid pack 43 to the second intermediate container 42b. Although the chemical liquid pack 43 is not under the gas pressure control exercised by the liquid delivery apparatus 11, as a result of the liquid delivery apparatus 11 decreasing the pressure of the second intermediate container 42b provided on the inflow side in this manner, it is possible to exercise the liquid delivery control over the PBS stored in the chemical liquid pack 43.

The location where the PBS flows from the chemical liquid pack 43 to the second intermediate container 42b corresponds to the liquid delivery path 8c in the first PBS rinse suction step. The liquid delivery path 8c includes the pressure loss member 6b before the second intermediate container 42b.

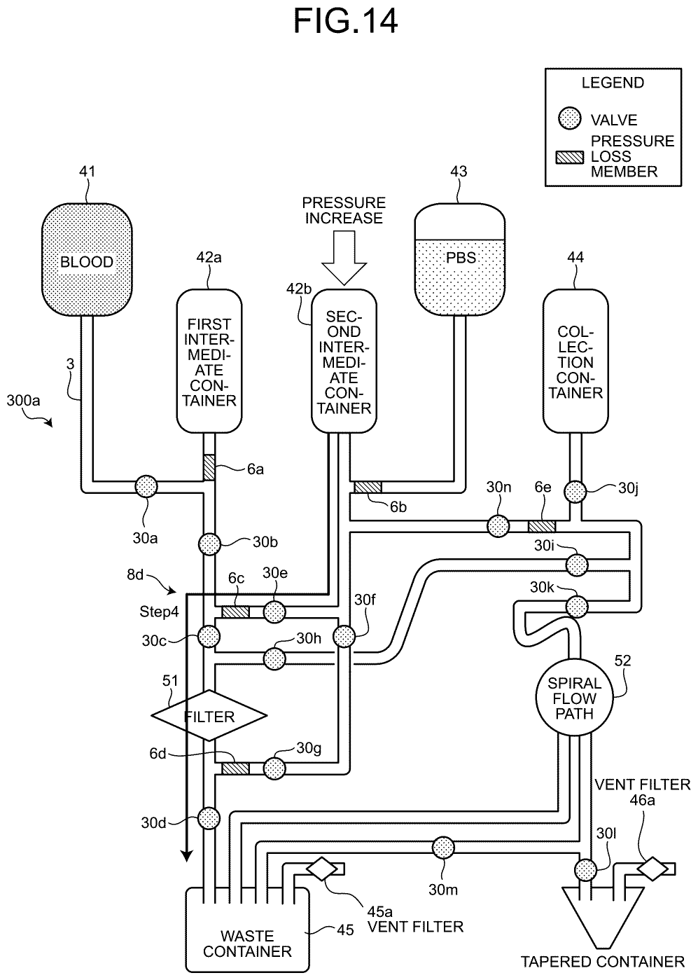

FIG. 14 is a diagram illustrating an example of a liquid delivery path 8d in the PBS rinse step according to the present embodiment. In the PBS rinse step, as a result of the liquid delivery apparatus 11 increasing the pressure of the second intermediate container 42b, the PBS is delivered from the second intermediate container 42b, via the filter 51, to the waste container 45. The location where the PBS flows from the second intermediate container 42b to the waste container 45 corresponds to the liquid delivery path 8d in the PBS rinse step.

Because the PBS rinse step is performed for the purpose of cleaning the filter 51 and the flow path 300a before the cell collecting step is performed at the subsequent stage, the liquid delivery path 8d includes, from the liquid delivery path 8b in the blood filtering step, all of the part that overlaps with the liquid delivery path in the cell collecting step performed at the subsequent stage.

The liquid delivery path 8d includes the pressure loss member 6c in a position that is on the upstream side of the filter 51 and that does not overlap with the liquid delivery path 8b in the blood filtering step. In other words, the liquid delivery path 8b and the liquid delivery path 8d are not sharing any pressure loss member 6 and include the pressure loss member 6a and the pressure loss member 6c respectively. The reason is that the magnitude of the pressure loss required in the liquid delivery path 8b is different from that required in the liquid delivery path 8d, because the speed required when the blood flows into the filter 51 in the blood filtering step is different from the speed required when the PBS flows into the filter 51 in the PBS rinse step, and because the blood and PBS have mutually-different viscosity and density levels. Furthermore, because the shapes of the liquid delivery path 8b and the liquid delivery path 8d themselves excluding the pressure loss members 6 are mutually different, the values of the pressure losses occurring in the liquid delivery path 8b and the liquid delivery path 8d are also mutually different. Providing the liquid delivery path 8b and the liquid delivery path 8d with the separate pressure loss members 6a and 6c, respectively, makes it possible to individually adjust the total pressure losses in the liquid delivery paths 8b and 8d.

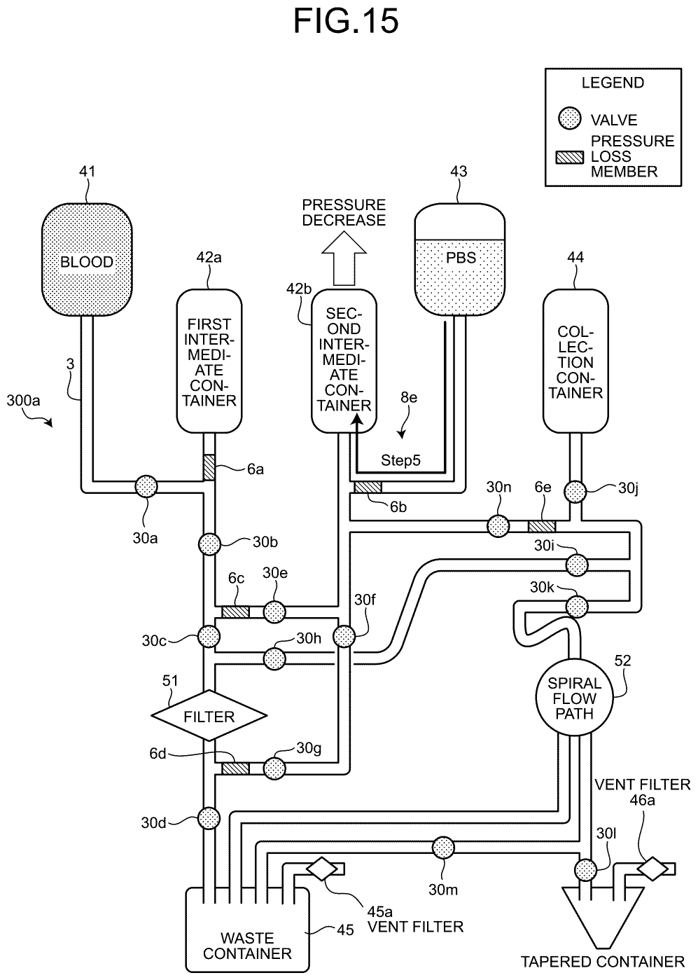

FIG. 15 is a diagram illustrating an example of a liquid delivery path 8e in the second PBS rinse suction step according to the present embodiment. The liquid delivery path 8e in the second PBS rinse suction step is the same as the liquid delivery path 8c in the first PBS rinse suction step.

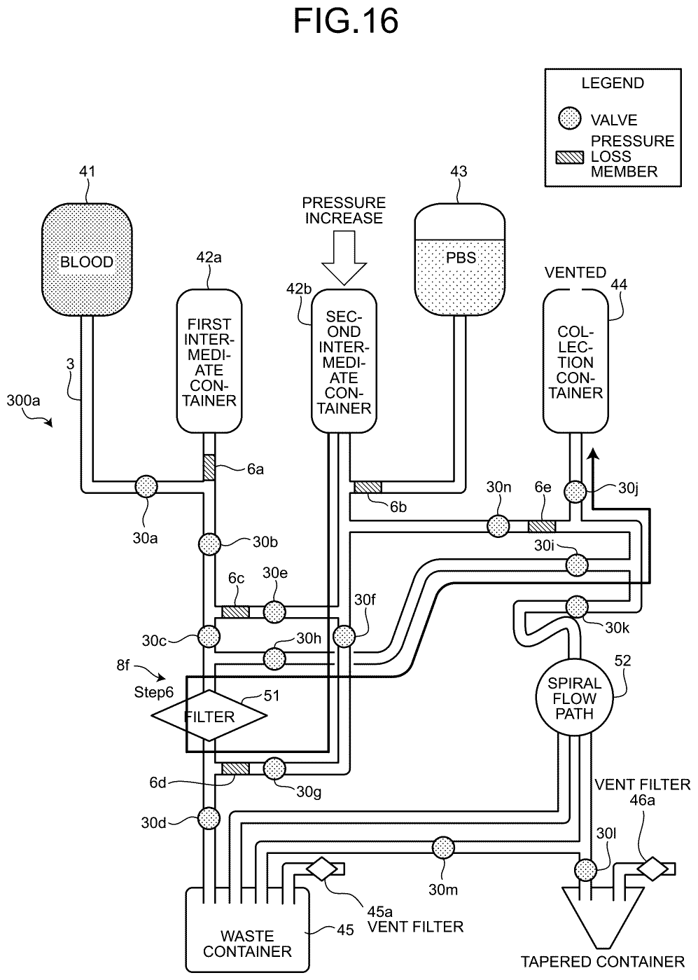

FIG. 16 is a diagram illustrating an example of a liquid delivery path 8f in the cell collecting step according to the present embodiment. In the cell collecting step, as a result of the liquid delivery apparatus 11 increasing the pressure of the second intermediate container 42b while the collection container 44 is vented, the PBS is delivered from the second intermediate container 42b, via the filter 51, to the collection container 44. Accordingly, the cells that have been rinsed are collected from the filter 51 and temporarily stored in the collection container 44. The location where the PBS flows from the second intermediate container 42b to the collection container 44 corresponds to the liquid delivery path 8f in the cell collecting step.

The liquid delivery path 8f includes the pressure loss member 6d provided in a position that is on the upstream side of the filter 51 and that does not overlap with the liquid delivery paths 8b and 8d. In other words, the liquid delivery path 8f does not share any pressure loss member 6 with the liquid delivery paths 8b and 8d and includes the unique pressure loss member 6d. As a result, the liquid delivery path 8f is able to adjust the total pressure loss in the liquid delivery path 8f, separately from the liquid delivery paths 8b and 8d.

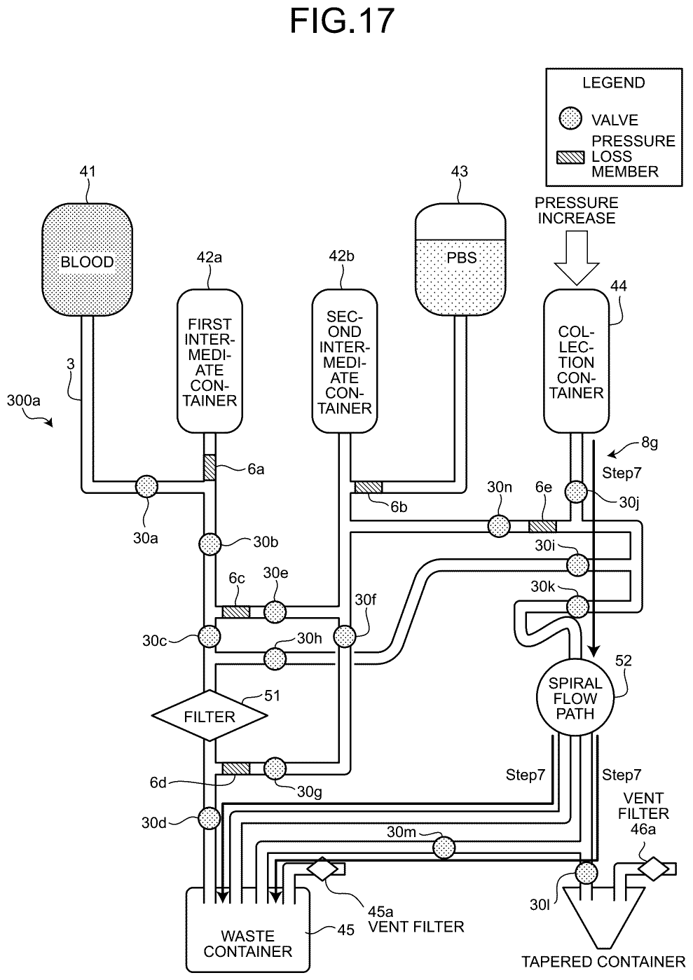

FIG. 17 is a diagram illustrating an example of a liquid delivery path 8g in the first classifying step according to the present embodiment. The first classifying step is a pre-processing process of the subsequent second classifying step. Until the classification stabilizes, all of the PBS that has flowed into the spiral flow path 52 is discharged into the waste container 45.

In the first classifying step, as a result of the liquid delivery apparatus 11 increasing the pressure of the collection container 44, the PBS is delivered from the collection container 44, via the spiral flow path 52, to the waste container 45. The location where the PBS flows from the collection container 44 to the waste container 45 corresponds to the liquid delivery path 8g in the first classifying step. The liquid delivery path 8g includes no pressure loss member 6.

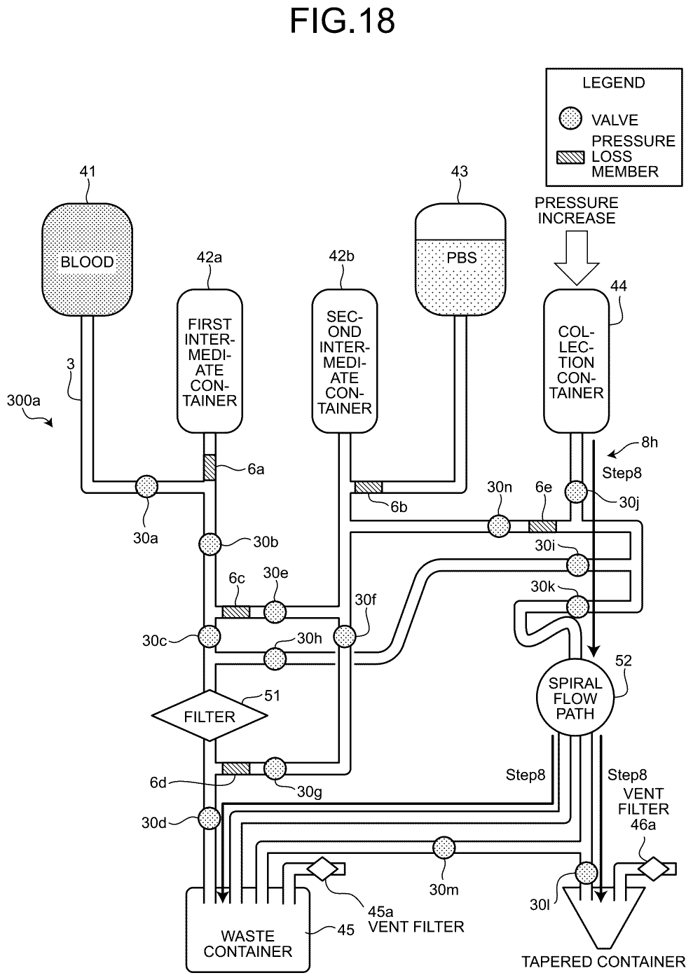

FIG. 18 is a diagram illustrating an example of a liquid delivery path 8h in the second classifying step according to the present embodiment. The second classifying step is a step performed after the classification has stabilized in the first classifying step.

In the second classifying step, as a result of the liquid delivery apparatus 11 increasing the pressure of the collection container 44, the PBS is delivered from the collection container 44, via the spiral flow path 52, into the waste container 45 and into the tapered container 46 separately. The cells collected in the collecting part 525 illustrated in FIG. 6 flow into the tapered container 46 through the liquid delivery path 8h. In contrast, the cells that have flowed into the discarding part 524 illustrated in FIG. 6 are discharged into the waste container 45 through the liquid delivery path 8h. The location where the PBS flows from the collection container 44 to the waste container 45 and the tapered container 46 corresponds to the liquid delivery path 8h in the second classifying step. The liquid delivery path 8h includes no pressure loss member 6.

Among the liquid delivery paths 8a to 8h in the abovementioned eight steps, each of the liquid delivery paths 8a to 8f, except for the liquid delivery paths 8g and 8h illustrated in FIGS. 17 and 18, includes one of the pressure loss members 6a to 6d. The reason why the maximum number of pressure loss members (6a to 6d) in each of the liquid delivery paths 8a to 8f is one is that the purpose is to reduce pulsation caused by an eddy occurring in the liquid due to changes in the inside diameter of the flow path 300a at the exits/entrances of the pressure loss members 6a to 6d. When the quantity of the pressure loss members 6 in the liquid delivery paths 8a to 8f increases, the pulsation is expected to be combined or amplified. Because an increase in the pulsation affects precision levels of the flow rates, it is desirable to keep small the quantity of the pressure loss members 6 for each of the liquid delivery paths 8a and 8f.

Further, the liquid delivery paths 8g and 8h include no pressure loss member 6. The reason is that the spiral flow path 52 included in the liquid delivery paths 8g and 8h includes the micro flow path 521 having a smaller inside diameter than that of each of the pipes 3, and the spiral flow path 52 therefore has a large pressure loss. In other words, because the liquid delivery paths 8g and 8h have large pressure losses even without having pressure loss members 6, no pressure loss member 6 is required.

The liquid delivery paths 8a to 8f are examples of a first liquid delivery path in the flow path 300a. The liquid delivery paths 8g and 8h are examples of a second liquid delivery path in the flow path 300a. In the following sections, when not being particularly distinguished individually, the liquid delivery paths 8a to 8h will simply be referred to as liquid delivery paths 8.

The liquid delivery paths 8 described with reference to FIGS. 11 to 18 are merely examples, and possible liquid delivery paths in the flow path 300a are not limited to these examples.

Next, the pressure loss members 6 according to the present embodiment will be explained in detail.

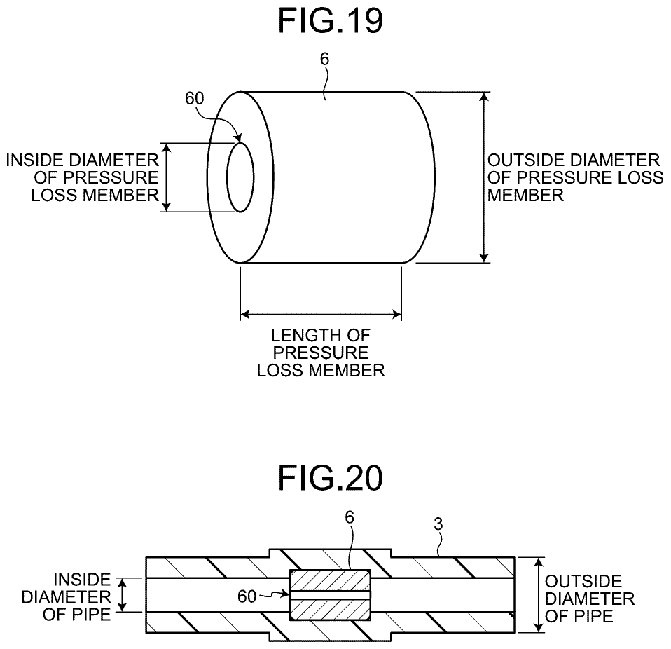

FIG. 19 is a drawing illustrating an example of the pressure loss members 6 according to the present embodiment. FIG. 20 is a drawing illustrating an example of a state in which a pressure loss member 6 according to the present embodiment is placed in a pipe 3. As illustrated in FIG. 20, the pressure loss member 6 is provided while being inserted through the pipe 3.

As illustrated in FIGS. 19 and 20, the pressure loss member 6 according to the present embodiment is a tubu having a through hole 60. It is desirable to select materials of the pressure loss member 6, while keeping in mind that the iPS cells are to be produced and returned to a human body. For example, it is possible to use zirconia, polyether ether ketone (PEEK) resin, stainless steel, titanium, a cobalt chrome alloy, polyethylene, nylon, silicone, Teflon (a registered trademark), or polyurethane. Among these, from the viewpoint of easily guaranteeing precision levels for dimensions of the inside diameter and the outside diameter, it is desirable to use one selected from among zirconia, polyether ether ketone (PEEK) resin, stainless steel, titanium, and a cobalt chrome alloy. In particular, to precisely control the pressure losses, it is desirable to realize the capability to design and generate the dimensions of the inside diameters of the pressure loss members 6 at the level ranging from 1 micrometer to 9 micrometers. For this reason, among the materials listed above, it is particularly preferable to use zirconia, which can be processed especially with a high level of precision. Examples of methods that can be used for processing zirconia include injection molding and hole drilling.

The diameter of the through hole 60 in the pressure loss member 6, i.e., the inside diameter of the pressure loss member 6, is smaller than the inside diameter of each of the pipes 3. For this reason, in the liquid delivery paths 8 of the flow path 300a, the locations where the pressure loss members 6 are inserted in the pipes 3 are each a location where the hole for passing the liquid is smaller than the holes in the locations before and after.

In this situation, because an inner hollow part of each of the pipes 3 is the part through which the liquid flows, a cross-sectional area of the inner hollow part of each of the pipes 3 will be referred to as a cross-sectional area of the liquid delivery path 8. In contrast, as for the locations in the pipes 3 where the pressure loss members 6 are placed, because the through hole 60 in the pressure loss member 60 is the part through which the liquid flows, a cross-sectional area of the through hole 60 in the pressure loss member 6 will be referred to as a cross-sectional area of the liquid delivery path 8. Accordingly, in other words, the pressure loss member 6 is structured so as to have a smaller cross-sectional area than cross-sectional areas before and after the pressure loss member 6 in the liquid delivery path 8. Further, while the liquid is flowing through the flow path 300a, a cross-sectional area of the liquid in the locations having the pressure loss members 6 in the liquid delivery paths 8 is smaller than cross-sectional areas of the liquid in the pipes 3 positioned before and after each pressure loss member 6.

As explained above, for the pipes 3, silicone tubes having an outside diameter of 3 mm and an inside diameter of 1 mm may be used, in an example. In that situation, the inside diameter of each of the pressure loss members 6 is smaller than 1 mm. Depending on the inside diameters and the lengths of the pressure loss members 6, the magnitudes of the pressure losses generated by the pressure loss members 6 will vary. In the present embodiment, to control the gas pressure of the flow path 300a by using the generic regulator, the inside diameters and the lengths of the pressure loss members 6 are determined so that the total pressure loss of the entire liquid delivery path 8 is kept within the range of 30 kPa to 260 kPa.

Next, a method for calculating the pressure losses in the liquid delivery paths 8 will be explained. As for the values of the pressure losses in the liquid delivery paths 8, mutually-different formulae are used in the calculations for the pipes and component parts and for the locations where the inside diameter radically increases or decreases. Examples of the component parts include joints used at the connection parts of the pipes 3. Examples of the locations where the diameter radically increases or decreases include the pressure loss members 6 and the spiral flow path 52.

$$PRESSURE LOSS Δ p = \frac{128 μL}{π {D}^{4}} V \qquad ( 1 )$$

μ: FLUID VISCOSITY [Pa·s]
L: PIPE LENGTH [m]
D: PIPE INSIDE DIAMETER [m]
V: FLOW RATE [m³/s]

Expression (1) presented above is a formula for calculating a pressure loss of a pipe or a component part. In the expression, μ denotes fluid viscosity (Pa·s); L denotes a pipe length (m); D denotes a pipe inside diameter (m); and V denotes a flow rate (m³/s).

$$COMPONENT PART LENGTH EQUIVALENT TO PIPE LENGTH Le = nD \qquad ( 2 )$$

n: EQUIVALENT LENGTH COEFFICIENT

TABLE 3

| | Value n for component part |
|---|---|
| T-shaped straight flow | 40 |
| T- shaped perpendicular turn | 80 |

Expression (2) presented above is a formula for converting the length of a component part into an equivalent length of the pipe 3, to calculate a pressure loss of a component part for the pipes 3 and the like, by using Expression (1). In the expression, n denotes an equivalent length coefficient. The equivalent length Le is used by being assigned to L in Expression (1). Further, Table 3 presented above indicates the value of n for each of the different types of joints used in the connection parts of the pipes 3.

$$PRESSURE LOSS Δ p = K \frac{8 ρ}{{π}^{2} {D}^{4}} {V}^{2} \qquad ( 3 )$$

ρ: FLUID DENSITY [kg/m³]

Expression (3) presented above is a formula for calculating a pressure loss in a location where the inside diameter of the flow path 300a is radically enlarged or reduced. In the expression, ρ denotes fluid density (kg/m³), whereas K denotes a loss coefficient.

$$Kc = - 0.5 \frac{{A}_{2}}{{A}_{1}} + 0.5 \qquad ( 4 )$$

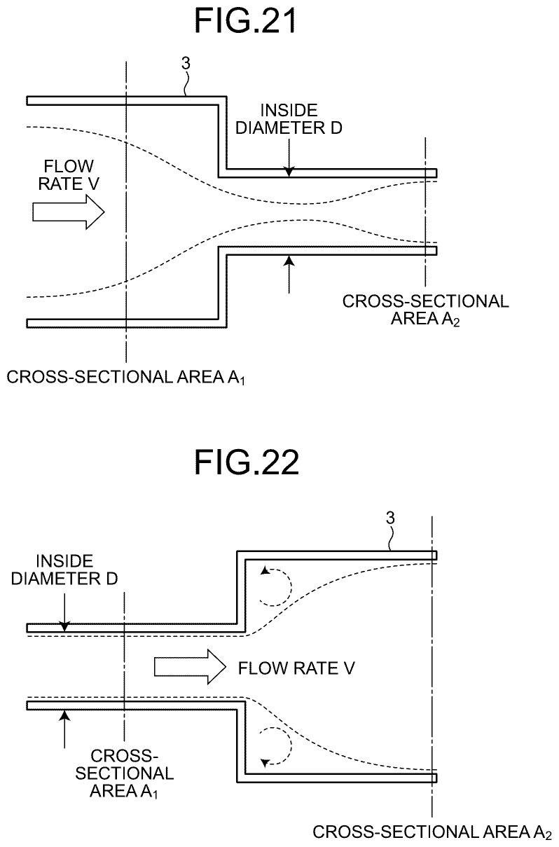

Expression (4) presented above is a formula for calculating a loss coefficient Kc of an inside diameter reduced part. Further, FIG. 21 is a drawing illustrating an example of a reduced part of the pipe 3 according to the present embodiment. As illustrated in FIG. 21, a pipe cross-sectional area A₁ is a cross-sectional area of the inner hollow part of the pipe 3 in the part where the inside diameter of the pipe 3 is not reduced. A pipe cross-sectional area A₂ is a cross-sectional area of the inner hollow part of the pipe 3 in the part where the inside diameter of the pipe 3 is reduced.

The loss coefficient Kc is calculated by assigning a pipe cross-sectional area ratio A₂/A₁ to Expression (4). The part where the inside diameter of the pipe 3 is reduced may be, for example, an entrance part of any of the pressure loss members 6. However, also for other parts where the inside diameter of the pipe 3 is reduced for other reasons, it is possible to calculate a pressure loss by using Expression (3) and Expression (4).

$$Ke = {( 1 - \frac{{A}_{1}}{{A}_{2}} )}^{2} \qquad ( 5 )$$

Expression (5) presented above is a formula for calculating a loss coefficient Ke of an inside diameter enlarged part. Further, FIG. 22 is a drawing illustrating an example of an enlarged part of the pipe 3 according to the present embodiment. In the calculation of the loss coefficient Ke of the enlarged part of the pipe 3, as illustrated in FIG. 22, the pipe cross-sectional area A₁ is a cross-sectional area of the inner hollow part of the pipe 3 in the part where the inside diameter of the pipe 3 is not enlarged (i.e., the part where the inside diameter of the pipe 3 is reduced). The pipe cross-sectional area A₂ is a cross-sectional area of the inner hollow part of the pipe 3 in the part where the inside diameter of the pipe 3 is enlarged (i.e., the part where the inside diameter of the pipe 3 is not reduced). The loss coefficient Ke is calculated by assigning the pipe cross-sectional area ratio A₁/A₂ to Expression (5). The part where the inside diameter of the pipe 3 is enlarged may be, for example, an exit part of any of the pressure loss members 6. However, also for other parts where the inside diameter of the pipe 3 is enlarged for other reasons, it is possible to calculate a pressure loss by using Expression (3) and Expression (5). In addition, a pressure loss on the inside of any of the pressure loss members 6 is calculated by using Expression (1).

The reduction and the enlargement of the parts where the liquid flows in the pipe 3 illustrated in FIGS. 21 and 22 may be reduction and enlargement realized by the pressure loss member 6, for example; however, illustration of the pressure loss member 6 is omitted from FIGS. 21 and 22. In FIGS. 21 and 22, the broken lines within the pipe 3 indicate the flow of the liquid in the pipe 3. As illustrated in FIGS. 21 and 22, because pulsation occurs in the liquid before and after the position in which the part where the liquid flows in the pipe 3 is reduced and enlarged, the speed and the flow rate of the liquid fluctuates. The larger the inside diameter difference is between the normal part of the pipe 3 and the reduced or enlarged part, the larger is the pulsation. Also, the higher the speed and/or the flow rate of the liquid is, the larger is the pulsation.

TABLE 4

| | |
|---|---|
| Flow path length | 600 |
| [mm] | |
| T-shaped straight flow | 3 |
| [Location] | |
| T-shaped perpendicular turn | 4 |
| [Location] | |
| T-shaped converted flow path length | 440 |
| [mm] | |
| Total flow path length | 1040 |
| [mm] | |
| Flow path pressure loss | 9.85 |
| [kPa] | |
| Second intermediate container inside | 10 |
| diameter | |
| [mm] | |
| Second intermediate container exit | 0.025 |
| pressure loss | |
| [kPa] | |
| Collection container inside diameter | 10 |
| [mm] | |
| Collection container entrance | 0.050 |
| pressure loss | |
| [kPa] | |
| Filter pressure loss | 0.1 |
| [kPa] | |
| Total pressure loss | 10.03 |
| [kPa] | |

Regarding the liquid delivery path 8f in the cell collecting step explained with reference to FIG. 16, Table 4 indicates a breakdown and a total of the pressure losses caused by the structure excluding the pressure loss members 6. As a premise, it is assumed that the liquid flowing through the liquid delivery path 8f is physiological saline and that the flow rate per unit period is 15 mL/min. As presented in Table 4, when no pressure loss member is included, the pressure loss of the liquid delivery path 8f is as small as 10.03 kPa in total. Accordingly, there is a large change amount in the flow rate of the liquid, in response to fluctuation of the gas pressure, which means that the liquid delivery control has a high degree of difficulty. Further, the total pressure loss of the liquid delivery paths 8g and 8h including the spiral flow path 52 illustrated in FIGS. 17 and 18 exceeds at least 250 kPa. Consequently, the fact that there is a large pressure loss difference between the liquid delivery path 8f and the liquid delivery path 8g or 8h is also a factor that raises the degree of difficulty of the liquid delivery control.

To cope with the circumstances described above, the inside diameter and the length of the pressure loss member 6d are determined so that the total pressure loss of the entire liquid delivery path 8f is kept within the range of 30 kPa to 260 kPa.

TABLE 5

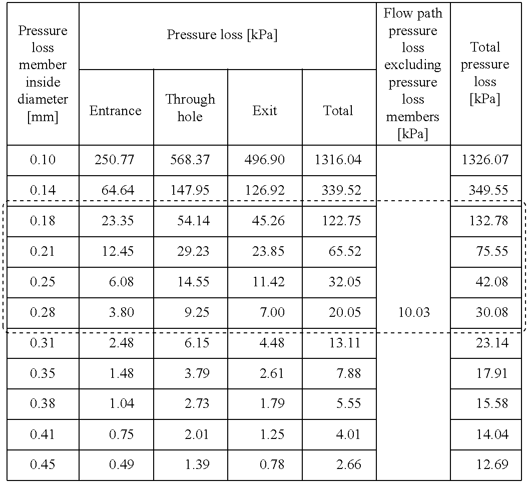

Table 5 indicates pressure loss values for the mutually-different inside diameters, on the assumption that the outside diameter of the pressure loss member 6d is 1.6 mm, while the length thereof is 6 mm. As a premise, it is assumed that the liquid flowing into the pressure loss member 6d is physiological saline and that the flow rate per unit period is 15 mL/min. As indicated in the section enclosed by the broken line in Table 5, when the inside diameter of the pressure loss member 6d is in the range of 0.28 to 0.18, the total pressure loss of the entire liquid delivery path 8f is kept within the range of 30 kPa to 260 kPa.

Generally speaking, it is easier to control the increasing and the decreasing of the gas pressure, when the total pressure loss of the entire liquid delivery path 8f is larger; however, when the total pressure loss is too large, large loads are imposed on the containers 4 and the joints structuring the flow path 300a. In addition, the larger the difference is between the inside diameter of the pressure loss member 6d and the inside diameter of the pipe 3, the larger is the pulsation to be caused. For these reasons, the pressure loss of the pressure loss member 6d and the total pressure loss of the entire liquid delivery path 8f shall be determined while being balanced against a level of precision required of the liquid delivery control.

Although Tables 4 and 5 use the liquid delivery path 8f as an example, it is also possible to use the same method for the liquid delivery paths 8a to Be in the flow path 300a, to calculate the lengths and the inside diameters of the pressure loss members 6a to 6c that are capable of keeping the total pressure losses within the appropriate range.

The pressure loss values presented above are merely examples. The total pressure loss of any of the liquid delivery paths 8 as well as the inside diameters and the lengths of the pressure loss members 6 are to be changed, in accordance with capabilities of the regulator and the configuration of the flow path 300a included in the liquid delivery apparatus 11.

As explained above, the cartridge 10 of the present embodiment includes the pressure loss members 6a to 6e provided in the liquid delivery paths 8 in the flow path 300a and structured so that the cross-sectional area of the liquid delivered through the flow path 300a is smaller than the cross-sectional areas before and after each pressure loss member. Accordingly, because it is possible to adjust the total pressure loss of the entire liquid delivery path 8, it is possible to ensure that the pressure loss differences among the plurality of liquid or gas delivery processes are kept within the appropriate range.

More specifically, in the cartridge 10 according to the present embodiment, the location having the pressure loss member 6 has a larger pressure loss value than that in each of the pipes 3 positioned before and after the pressure loss member 6. It is therefore possible to increase the pressure loss of the entire liquid delivery path 8.

Further, the cartridge 10 according to the present embodiment includes the pressure chambers 200 each configured to receive, from the external source, the motive power for delivering the liquid. Thus, while the closed system in the cartridge 10 is maintained, it is possible to control the deliveries of the liquid in the flow paths 300 and 300a.

The flow path 300a in the cartridge 10 according to the present embodiment includes the pipes 3 each having the hole through which the liquid flows, while the pressure loss members 6 are provided while being inserted through the pipes 3. Consequently, by using the cartridge 10 of the present embodiment, it is possible to easily position the pressure loss members 6 in accordance with the configurations of the liquid delivery paths 8.

The flow path 300a in the cartridge 10 of the present embodiment includes the spiral flow path 52 configured to sort out the objects in the liquid by using the flowing action. Further, the flow path 300a includes: the liquid delivery paths 8a to 8f that do not include the spiral flow path 52, but include the pressure loss members 6; and the liquid delivery paths 8g and 8h that include the spiral flow path 52, but include no pressure loss member 6. Accordingly, in the cartridge 10 according to the present embodiment, it is possible to increase, by using the pressure loss members 6, the pressure losses of only the liquid delivery paths 8a to 8f that do not include the spiral flow path 52, without increasing the pressure losses of the liquid delivery paths 8g and 8h including the spiral flow path 52 and having a large total pressure loss.

The flow path 300a in the cartridge 10 of the present embodiment includes the filter 51 configured to sort out the objects from the liquid. Further, the flow path 300a includes the pressure loss member 6 provided on the upstream side of the filter 51 in the flow path 300a. Accordingly, by using the cartridge 10 according to the present embodiment, it is possible to properly adjust the speed of the liquid flowing into the filter 51.

The flow path 300a in the cartridge 10 of the present embodiment includes the plurality of liquid delivery paths 8 through which the liquid flows, while each of the plurality of liquid delivery paths 8 includes at most one pressure loss member 6. Consequently, by using the cartridge 10 of the present embodiment, it is possible to reduce the pulsation that may be caused by the pressure loss member 6, while adjusting the total pressure loss of the entire liquid delivery paths 8.

The cell production apparatus 1 according to the present embodiment includes the cartridge 10 and the liquid delivery apparatus 11. Further, the liquid delivery apparatus 11 is configured to transmit the liquid delivery drive to the pressure chambers 200 in the cartridge 10, from the outside of the cartridge 10. Consequently, by using the cell production apparatus 1 according to the present embodiment, it is possible to control the liquid deliveries while maintaining the closedness of the flow path 300a in the cartridge 10. In addition, in the cell production apparatus 1 according to the present embodiment, because the single liquid delivery apparatus 11 is used in common to the plurality of liquid delivery paths 8, advantageous effects are achieved where the configuration of the apparatus is simplified and costs are reduced, as compared to a configuration in which a liquid delivery pump or the like is provided for each of the liquid delivery paths 8.

In the present embodiment, from among the flow paths 300 included in the cartridge 10, the flow path 300a corresponding to the blood introducing process and the blood corpuscle separating process was explained as an example; however, it is possible to use the pressure loss members 6 not only in the blood introducing process or the blood corpuscle separating process, but also in other various liquid delivery paths in the cell production apparatus 1.

Further, besides the cell production apparatus 1, the pressure loss members 6 are also applicable to various flow paths over which liquid delivery control using pressure is exercised.

The shapes, the configurations, and the pressure loss values of the flow paths 300 and 300a explained in the present embodiment are all merely examples, and possible embodiments are not limited to the above examples. Further, although the cell production apparatus 1 and the cartridge 10 are configured to generate the iPS cells in the present embodiment, the cell production apparatus 1 and the cartridge 10 may be apparatuses configured to produce other types of cells.

### First Modification Example

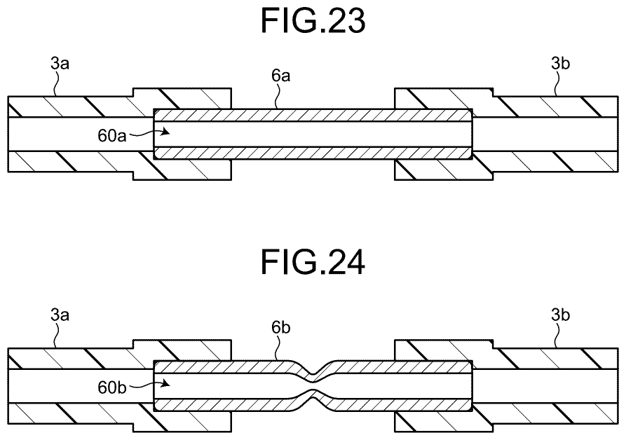

Possible configurations of the pressure loss members 6 are not limited to the configurations described in the above embodiment. FIG. 23 is a drawing illustrating an example of the pressure loss member 6a according to a first modification example. The pressure loss member 6a is a tubular needle pipe having a narrow through hole 60a. The pressure loss member 6a is positioned so as to connect the pipes 3 together. More specifically, one end of the pressure loss member 6a is inserted in a pipe 3a, whereas the other end is inserted in another pipe 3b. The pipe 3a is an example of a first pipe in the present modification example. The pipe 3b is an example of a second pipe in the present modification example.

The diameter of the through hole 60a in the pressure loss member 6a, i.e., the inside diameter of the pressure loss member 6a is configured to be smaller than the inside diameters of the pipes 3a and 3b. Accordingly, the pressure loss member 6a is structured so that cross-sectional areas thereof are smaller than cross-sectional areas before and after the pressure loss member 6a in the liquid delivery path 8. Consequently, in the liquid delivery path, cross-sectional areas of the liquid in the location having the pressure loss member 6a are smaller than cross-sectional areas of the liquid in the pipes 3 positioned before and after the pressure loss member 6a. In addition, the pressure loss member 6a is structured to be thinner than the pressure loss members 6 described in the above embodiment. Accordingly, the inside diameter of the pressure loss member 6a is larger than the inside diameters of the pressure loss members 6. In the present modification example, by making the pressure loss member 6a longer than the pressure loss members 6, it is possible to generate a pressure loss equivalent to that of each of the pressure loss members 6.

### Second Modification Example

FIG. 24 is a drawing illustrating an example of the pressure loss member 6b according to a second modification example. The pressure loss member 6b is a pipe in which a part of a needle pipe is narrowed. The narrowed part of the pressure loss member 6b is formed by pinching the needle pipe, for example. Cross-sections of the pressure loss member 6b in the pinched location may be oval shapes, for example.

Similarly to the pressure loss member 6a, the pressure loss member 6b is provided so as to connect the pipes 3 together. One end of the pressure loss member 6b is inserted in the pipe 3a, whereas the other end is inserted in the other pipe 3b. In the pinched location, the inner hollow part through which the liquid flows is smaller than inner hollow parts of the pipes 3a and 3b. Accordingly, cross-sectional areas of a through hole 60b in the pressure loss member 6b are, at least partially, smaller than cross-sectional areas of the pipes 3a and 3b. Further, in the liquid delivery path, cross-sectional areas of the liquid in the location having the pressure loss member 6b are smaller than cross-sectional areas of the liquid in the pipes 3 positioned before and after the pressure loss member 6b.

In the present modification example, by pinching the needle pipe so that the inner hollow part thereof has desired cross-sectional areas, it is possible to adjust the pressure loss to be generated by the pressure loss member 6b.

### Third Modification Example

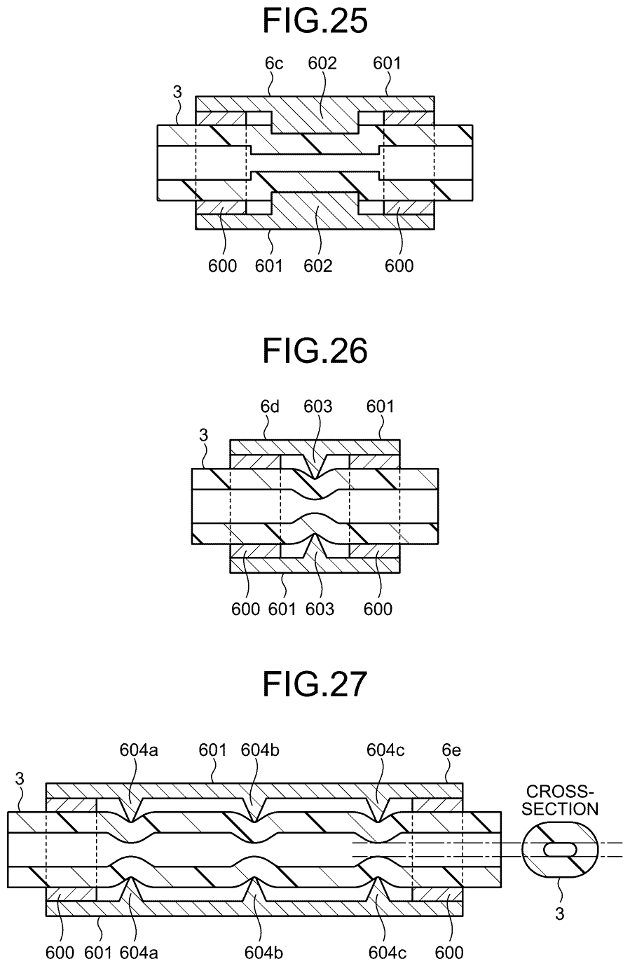

FIG. 25 is a drawing illustrating an example of the pressure loss member 6c according to a third modification example. In the third modification example, the pressure loss member 6c is a jig configured to crush the pipe 3 structured with a silicone tube, so as to realize desired cross-sectional areas. In the present modification example, the pressure loss member 6c is attached to the outside of the pipe 3. The pressure loss member 6c includes a surrounding part 600 configured to surround the pipe 3 and a pressing part 601 configured to press the pipe 3. The pressing part 601 includes projection parts 602 configured to press the pipe 3. The surrounding part 600 is configured to fix the pressing part 601 to the pipe 3. The surrounding part 600 may be referred to as a fixing part. The projection parts 602 of the present modification example have flat faces to be in contact with the pipe 3. The surrounding part 600 and the pressing part 601 may be structured by using metal, for example.

### Fourth Modification Example

FIG. 26 is a drawing illustrating an example of the pressure loss member 6d according to a fourth modification example. Similarly to the third modification example, the pressure loss member 6d according to the present modification example is a jig that is attached to the outside of the pipe 3 structured with a silicone tube and is configured to crush the pipe 3 so as to realize desired cross-sectional areas.

In the present modification example, the pressure loss member 6d includes the surrounding part 600 configured to surround the pipe 3 and the pressing part 601 configured to press the pipe 3. The pressing part 601 includes a projection part 603 configured to press the pipe 3. The projection part 603 in the present modification example is structured as a rib to be in contact with the pipe 3. As illustrated in FIG. 26, a cross-section of the projection part 603 may exhibit, in an example, triangles of which the corner that is in contact with the pipe 3 has an acute angle.

### Fifth Modification Example

FIG. 27 is a drawing illustrating an example of the pressure loss member 6e according to a fifth modification example. Similarly to the third and the fourth modification examples, the pressure loss member 6e according to the present modification example is a jig that is attached to the outside of the pipe 3 structured with a silicone tube and is configured to crush the pipe 3 so as to realize desired cross-sectional areas.

In the present modification example, the pressure loss member 6e includes the surrounding part 600 configured to surround the pipe 3 and the pressing part 601 configured to press the pipe 3. The pressing part 601 of the present modification example includes a plurality of projection parts 604a to 604c. Similarly to the fourth modification example, each of the projection parts 604a to 604c in the present modification example is structured as a rib to be in contact with the pipe 3.

The pressure loss members 6c to 6e in the third to the fifth modification examples are examples of a pressing part configured to reduce the cross-sectional areas of the hole in the pipe 3, by pressing the pipe 3. Cross-sections in the crushed location of the pipe 3 may be oval shapes, for example. In the liquid delivery path, cross-sectional areas of the liquid in the locations having the pressure loss members 6c to 6e are smaller than cross-sectional areas of the liquid in the pipes 3 positioned before and after the pressure loss members 6c to 6e.

When the pressure loss members 6c to 6e are the jigs attached to the outside of the pipe 3 as described in the third to the fifth modification examples, it is easy to attach, to detach, and to change the positions of, the pressure loss members 6c to 6e in the flow path 300.

### Sixth Modification Example

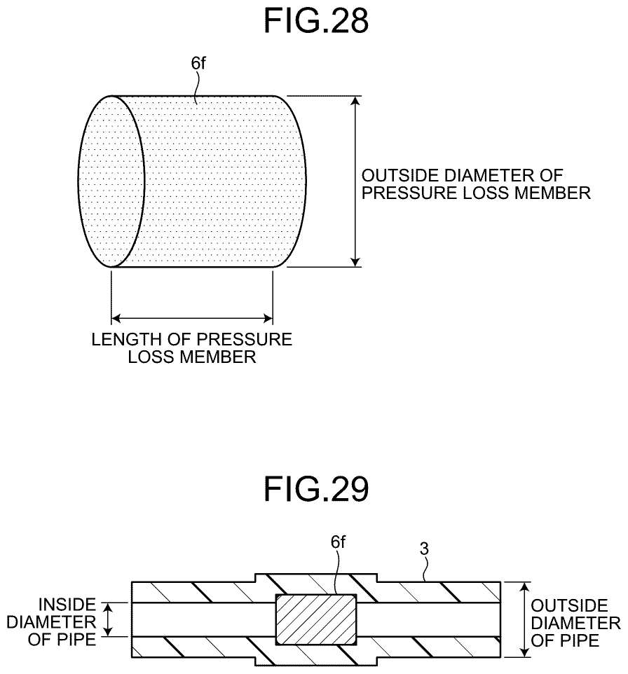

FIG. 28 is a drawing illustrating an example of a pressure loss member 6f according to a sixth modification example. FIG. 29 is a drawing illustrating an example of a state in which the pressure loss member 6f according to the sixth modification example is placed in the pipe 3. As illustrated in FIG. 29, the pressure loss member 6f is provided while being inserted through the pipe 3.

The pressure loss member 6f according to the present modification example is a porous molded member. The pressure loss member 6f has a plurality of holes (pores) which are smaller than the hole for the liquid flowing through the pipe 3 and through which the liquid and the cells in the liquid pass. The hole for the liquid flowing through the pipe 3 is an example of a first hole in the present modification example. The large number of small holes formed in the pressure loss member 6f being a porous body are examples of second holes in the present modification example.

For instance, when the outside diameter of the pipe 3 is approximately 3 mm, whereas the inside diameter thereof is approximately 1 mm, the outside diameter of the pressure loss member of may be approximately 1.6 mm, similarly to the pressure loss members 6 in the above embodiment. The pressure loss generated by the pressure loss member 6f varies in accordance with changes in the length of the pressure loss member 6f. Further, the magnitude of the pressure loss also varies depending on the sizes and the density (a total area of the pores) of the pores of the pressure loss member 6f. The length of the pressure loss member 6f and the size and the density of the pores of the pressure loss member of may be selected in accordance with the magnitude of the pressure loss required by the liquid delivery path being applied. In the present modification example, in the liquid delivery path, cross-sectional areas of the liquid in the location having the pressure loss member 6f are each a sum of cross-sectional areas of the pores on a certain cross-section of the pressure loss member 6f. Accordingly, in the present modification example also, in the liquid delivery path in the flow path 300a, cross-sectional areas of the liquid in the location having the pressure loss member 6f are smaller than cross-sectional areas of the liquid in the pipes 3 positioned before and after the pressure loss member 6f.

As explained above, it is possible to realize the pressure loss members 6 in various shapes. Accordingly, the shapes of the pressure loss members 6 may be selected as appropriate, in accordance with the property of the liquid flowing through the flow paths 300 and other requirements.

### Seventh Modification Example

In the above embodiment, it was stated that desirable materials of the pressure loss members 6 are materials capable of easily guaranteeing a precision level for the dimensions of the inside diameter and the outside diameter such as, more specifically, zirconia, PEEK resin, stainless steel, titanium, or a cobalt chrome alloy. As presented in Expression (1) above, the magnitude of the pressure loss fluctuates, depending on the value raised to the fourth power of a pipe inside diameter of the pressure loss member 6 or the like. It is therefore desirable that the dimensions of the pressure loss members 6 have high levels of precision. More specifically, it is desirable to ensure at least that errors in the dimensions of the pressure loss members 6 are 9 micrometers or smaller. In the above embodiment, it was stated that using zirconia, which can be processed with a high level of precision, as the material of the pressure loss members 6 is especially preferable. However, there are other means for guaranteeing the precision level of the pressure loss members 6.

For example, generally speaking, resin tubes that are commercially available have a possibility of having a design error of approximately 20 micrometers. For this reason, when the pressure loss members 6 are each a resin tube structured with PEEK resin or the like, it is desirable to have a measuring process and a secondary processing process before the resin tubes are incorporated into the cartridge 10, for the purpose of correcting product errors thereof.

In the measuring process, for example, an engineer may measure a generated pressure loss by actually causing gas to flow through the resin tube. Alternatively, an engineer may measure the dimensions of the resin tubes with a high level of precision by using various types of measuring tools and sensors.

In accordance with a result of the measuring process, the engineer or the like may perform the secondary processing process to correct an error that occurred at the time of shaping the resin tube and to realize a designed dimension. Examples of methods for performing the secondary processing process include: enlarging the inside diameter of the resin tube through shaving; and shortening the length by cutting the resin tube. When the resin tube resulting from the secondary processing process is incorporated as the pressure loss member 6 into the cartridge 10, it is possible to reduce an error that may be caused in the magnitude of the pressure loss. Further, the methods of the measuring process and the secondary processing process are also applicable to pressure loss members that are not resin tubes.

### Eighth Modification Example

In the above embodiment, the pressure loss members 6 are provided in the locations where the liquid flows through the flow paths 300; however, the pressure loss members 6 may be provided in locations where gas flows through the flow paths 300.

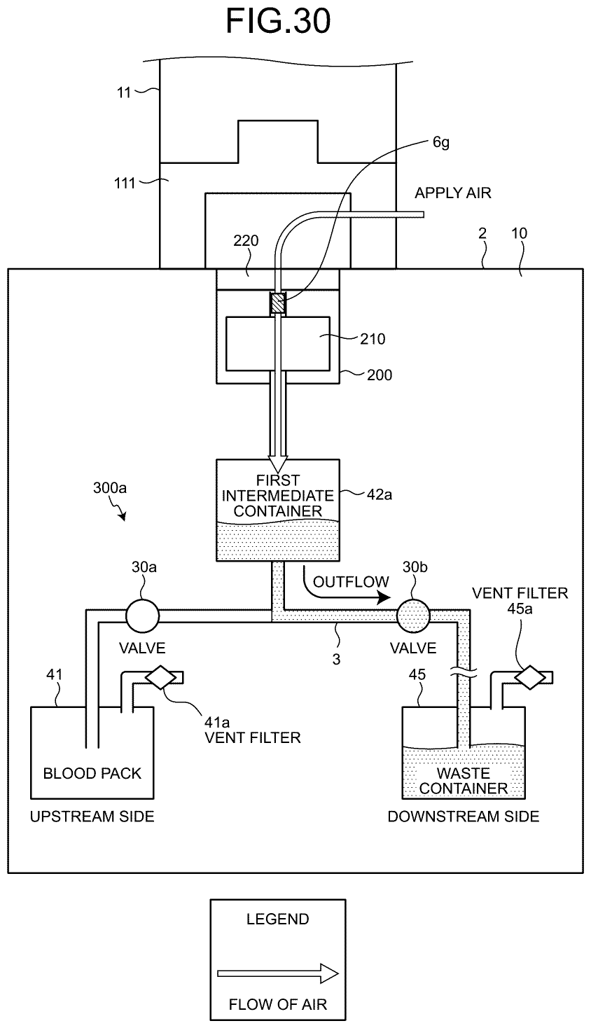

FIG. 30 is a diagram illustrating an example of an installation position of a pressure loss member 6g according to an eighth modification example. As illustrated in FIG. 30, the pressure loss member 6g may be installed on the downstream side of the sheet filter 220 in an example where the gas is applied to the cartridge 10. In FIG. 30, as an example, the pressure loss member 6g is provided in a position between the disc filter 210 and the sheet filter 220.

Further, although FIG. 30 illustrates the example of applying the gas, also in a situation where the gas is discharged, the same pressure loss member 6g may also be used for discharging the gas.

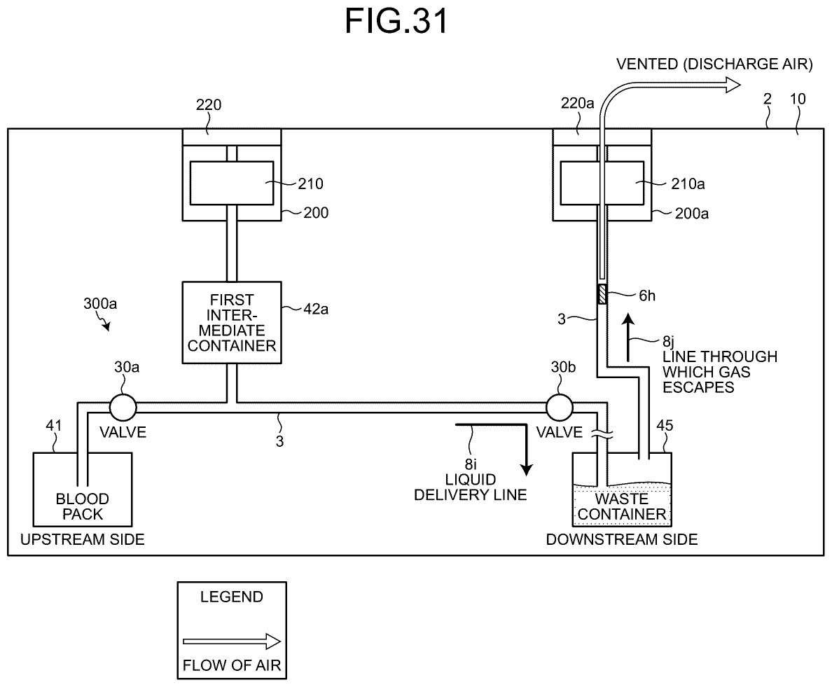

Further, FIG. 31 is a diagram illustrating another example of an installation position of a pressure loss member 6h according to the eighth modification example. In the example illustrated in FIG. 31, in the situation where gas is discharged from the flow path 300a through venting, a pressure chamber 200a, a disc filter 210a, and a sheet filter 220a are present on the downstream side of the waste container 45. In this configuration, the pressure loss member 6h may be provided in a certain position in the transport path where the gas flows on the downstream side of the waste container 45. In the example in FIG. 31, the pressure loss member 6h is provided in the pipe 3 connecting the waste container 45 to the pressure chamber 200a. In this situation, the path through which the gas escapes from the waste container 45 is different from the path through which the liquid flows into the waste container 45. For instance, in the example illustrated in FIG. 31, separately from a liquid delivery line 8i being the path through which the liquid flows from the first intermediate container 42a to the waste container 45, the pipe 3 provides a line 8j (a discharge path) through which the gas escapes from the waste container 45 to the pressure chamber 200a. The pressure loss member 6h is provided in the line 8j through which the gas escapes.

The installation positions of the pressure loss members 6g and 6h in FIGS. 30 and 31 are merely examples, and possible positions are not limited to these examples. It should be noted, however, that it is desirable when the pressure loss members 6g and 6h are not present in the path extending from the first intermediate container 42a to the waste container 45. If the pressure members 6g and 6h were present in the path extending from the first intermediate container 42a to the waste container 45, there would be a possibility that the liquid from the first intermediate container 42a passing through the pressure loss member might have a high flow rate, and a part of the liquid might remain in the pipe 3. In the situation where a part of the liquid remained in the pipe 3, it would be difficult to control the flow rate on the subsequent occasion when liquid is caused to flow.

As explained above, by providing the pressure loss members 6g and 6h in the certain locations where the gas flows in the transport paths within the flow paths 300, it is possible to adjust the pressure losses in the transport paths. Further, it is also acceptable to provide the pressure loss members 6 in both the locations where the gas flows and the locations where the liquid flows, within the transport paths. It is also acceptable to limit the quantity of the pressure loss members 6 in each transport path to one at most, including both the location where the gas flows and the location where the liquid flows.

Further, for the purpose of adjusting the pressure of the gas in the transport path, it is also acceptable to use a speed controller configured to adjust a motion speed of an air cylinder.

According to at least one aspect of the embodiments and the modification examples described above, it is possible to ensure that the differences in the pressure losses among the plurality of liquid or gas delivery processes are kept within the appropriate range.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A cell production cartridge including a flow path configured to deliver liquid or gas to be used for producing a cell, wherein
the flow path comprises a pressure loss generating part provided in a transport path for the liquid or the gas and structured so that a cross-sectional area of the liquid or the gas delivered through the flow path is smaller than cross-sectional areas before and after the pressure loss generating part.

2. The cell production cartridge according to claim 1, further comprising a drive receiving part configured to receive, from an external source, motive power for transporting the liquid or the gas.

3. The cell production cartridge according to claim 1 wherein
the flow path includes a pipe having a hole through which the liquid or the gas flows, and
the pressure loss generating part is provided while being inserted through the pipe.

4. The cell production cartridge according to claim 1, wherein
the flow path includes equipment configured to sort out an object in the liquid by using a flowing action, and
the flow path includes a first liquid delivery path that does not include the equipment but includes the pressure loss generating part and a second liquid delivery path that includes the equipment but does not include the pressure loss generating part.

5. The cell production cartridge according to claim 1, wherein
the flow path includes a filter configured to sort out an object from the liquid, and
the flow path includes the pressure loss generating part provided on an upstream side of the filter in the flow path.

6. The cell production cartridge according to claim 1, wherein
the flow path includes a plurality of liquid delivery paths through which the liquid flows, and
each of the plurality of liquid delivery paths includes the pressure loss generating part which is singular at most.

7. The cell production cartridge according to claim 3, wherein
the pressure loss generating part is a tubu having a through hole, and
a diameter of the through hole in the pressure loss generating part is smaller than an inside diameter of the pipe.

8. The cell production cartridge according to claim 1, wherein
the flow path includes a first pipe and a second pipe through which the liquid or the gas flows,
the pressure loss generating part is a tubu having a through hole,
a cross-sectional area of the through hole in the pressure loss generating part is, at least partially, smaller than a cross-sectional area of an inner hollow part of each of the first and the second pipes, and
one end of the pressure loss generating part is inserted in the first pipe, whereas the other end thereof is inserted in the second pipe.

9. The cell production cartridge according to claim 1, wherein
the flow path includes a pipe having a hole through which the liquid or the gas flows,
the pressure loss generating part is a pressing part configured to reduce a cross-sectional area of the hole in the pipe, by pressing the pipe.

10. The cell production cartridge according to claim 1, wherein
the flow path includes a pipe having a first hole through which the liquid or the gas flows, and
the pressure loss generating part is a porous body having a plurality of second holes which are each smaller than the first hole and through which the liquid or the gas passes.

11. The cell production cartridge according to claim 1, wherein the pressure loss generating part is structured by using one selected from among: zirconia, polyether ether ketone (PEEK) resin, stainless steel, titanium, and a cobalt chrome alloy.

12. The cell production cartridge according to claim 1, wherein
the flow path includes the transport path through which the gas flows, and
the transport path through which the gas flows includes the pressure loss generating part.

13. A cell production apparatus including a cell production cartridge and a liquid delivery apparatus, wherein
the cell production cartridge comprises:
a flow path configured to deliver liquid or gas to be used for producing a cell; and
a drive receiving part configured to receive, from an external source, motive power for delivering the liquid or the gas,
the flow path includes a pressure loss generating part provided in a transport path for the liquid or the gas and structured so that a cross-sectional area of the liquid or the gas delivered through the flow path is smaller than cross-sectional areas before and after the pressure loss generating part, and
the liquid delivery apparatus is configured to transmit a liquid delivery drive to the drive receiving part, from an outside of the cell production cartridge.

14. The cell production apparatus according to claim 13, wherein the cell production cartridge further comprises a drive receiving part configured to receive, from an external source, motive power for transporting the liquid or the gas.

15. The cell production apparatus according to claim 13, wherein
the flow path includes a pipe having a hole through which the liquid or the gas flows, and
the pressure loss generating part is provided while being inserted through the pipe.

16. The cell production apparatus according to claim 13, wherein
the flow path includes equipment configured to sort out an object in the liquid by using a flowing action, and
the flow path includes a first liquid delivery path that does not include the equipment but includes the pressure loss generating part and a second liquid delivery path that includes the equipment but does not include the pressure loss generating part.

17. The cell production apparatus according to claim 13, wherein
the flow path includes a filter configured to sort out an object from the liquid, and
the flow path includes the pressure loss generating part provided on an upstream side of the filter in the flow path.

18. The cell production apparatus according to claim 13, wherein
the flow path includes a plurality of liquid delivery paths through which the liquid flows, and
each of the plurality of liquid delivery paths includes the pressure loss generating part which is singular at most.

19. The cell production apparatus according to claim 15, wherein
the pressure loss generating part is a tubu having a through hole, and
a diameter of the through hole in the pressure loss generating part is smaller than an inside diameter of the pipe.

20. The cell production apparatus according to claim 13, wherein
the flow path includes the transport path through which the gas flows, and
the transport path through which the gas flows includes the pressure loss generating part.
